(19) United States
(12) Patent Application Publication
(10) Pub. No.: US 2024/0245692 A1
(43) Pub. Date: Jul. 25, 2024

(54) PHARMACEUTICAL COMPOSITION FOR PREVENTING OR TREATING RON-MUTATION-ASSOCIATED BILIARY TRACT CANCER

(71) Applicant: WELLMARKER BIO CO., LTD., Seoul (KR)

(72) Inventors: Seung-Mi KIM, Seoul (KR); Yeon-Seoung CHOI, Seoul (KR); Ha-Neul LEE, Seoul (KR); Ji-Hyun GO, Seoul (KR); Eun-Hee KO, Seoul (KR); Yu-Geun JI, Seoul (KR)

(73) Assignee: WELLMARKER BIO CO., LTD., Seoul (KR)

(21) Appl. No.: 18/289,697

(22) PCT Filed: May 3, 2022
(86) PCT No.: PCT/KR2022/006358

§ 371 Date: Nov. 6, 2023

(30) Foreign Application Priority Data
May 7, 2021 (KR) 10-2021-0059500

(51) Int. Cl. A61K 31/5377; A61K 31/444; A61K 31/4709; A61K 31/496; A61P 35/00

(52) U.S. Cl. CPC A61K 31/5377; A61K 31/444; A61K 31/4709; A61K 31/496; A61P 35/00

(57) ABSTRACT

A pharmaceutical composition containing a compound of Formula 1 or Formula 2 below or a pharmaceutically acceptable salt thereof. The composition is suitable for preventing or treating RON-mutation-associated biliary tract cancer. The pharmaceutical composition can be applied to a biliary tract cancer patient having a RON mutation. Particularly, the pharmaceutical composition can be effectively used for treating a biliary tract cancer patient who is resistant to cetuximab, which is conventionally used for cancer treatment, and who has mutation at RONA 155, RONΔ160 or RONΔ165.

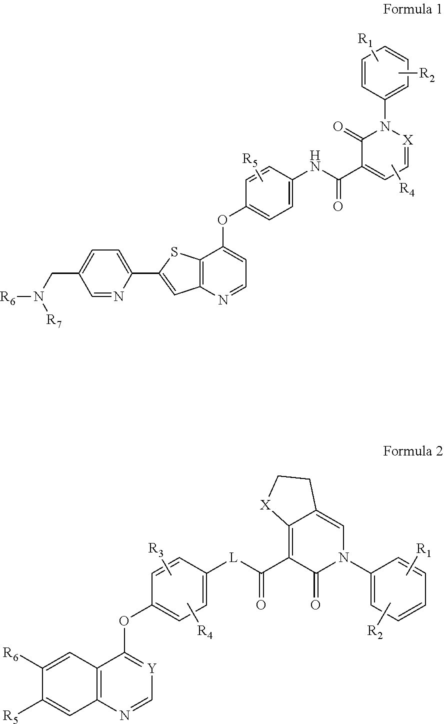

### TECHNICAL FIELD

The present invention relates to a pharmaceutical composition for preventing or treating cholangiocarcinoma associated with RON mutation, and a method for preventing or treating cholangiocarcinoma using the same.

### BACKGROUND ART

Cholangiocarcinoma is cancer that occurs in the bile duct which is a path that transports bile produced by the liver to the duodenum; and cholangiocarcinoma is largely divided into intrahepatic cholangiocarcinoma and extrahepatic cholangiocarcinoma depending on its location. In the United States, the incidence of cholangiocarcinoma is about 0.01% to 0.45% of all cancer patients; however, the incidence is higher in Asian countries and the incidence is increasing in countries such as North America, Europe, and Australia. According to the data from the Korea Central Cancer Registry published in 2020, cholangiocarcinoma accounted for about 2% of all cancer cases in 2018. Cholangiocarcinoma occurs frequently in people in their 50s to 70s. To date, the exact mechanism of cholangiocarcinoma is not known, and it is only known that environmental factors and genetic factors are involved in a complex way in cholangiocarcinoma.

The typical symptoms of cholangiocarcinoma include jaundice. When jaundice appears, the skin and whites of the eyes turn yellow, brown urine and grayish-white stool are excreted, and itchy skin occurs. However, jaundice appears when the cholangiocarcinoma has been advanced to some extent, and is often painless. For cholangiocarcinoma, in the early stages, symptoms of jaundice are rarely observed and no other symptoms are present, which makes it very difficult to detect the cholangiocarcinoma early. Although the incidence of cholangiocarcinoma is lower than that of other cancers, the prognosis is poor on average due to difficulty in early diagnosis and easy metastasis to nearby organs or lymph nodes.

For treatment methods for cholangiocarcinoma, radical resection, which removes all tissues related to the lesion, is primarily performed; however, only about 40% to 50% of patients are eligible for surgery. In cases where radical resection is not possible, a combination chemotherapy that combines gemcitabine and cisplatin and a combination chemotherapy that combines other anticancer agents such as capecitabine and oxaliplatin are tried. However, given that the survival rate of patients with cholangiocarcinoma according to the current treatment methods is less than 5%, there is a need to develop a more effective therapeutic technique.

In this regard, clinical trials using antibodies for targeted therapy such as panitumumab and cetuximab have been conducted on cholangiocarcinoma. However, limitations have been pointed out regarding therapeutic effects of panitumumab and cetuximab that in a case where epidermal growth factor receptor (EGFR) is mutated in cholangiocarcinoma, resistance to EGFR-targeting drugs occurs.

Resistance to EGFR-targeted therapeutics may be associated with RON (recepteur d'origine nantais) mutation. RON (recepteur d'origine nantais) refers to a protein receptor belonging to the c-MET family, and is a receptor for macrophage-stimulating protein (MSP) that is a serum protein, is secreted by the liver, and regulates the action of macrophages (Zhou Y Q et al., Oncogene 2003, 22(2): 186-197). The activity of RON plays an important function in the development, progression, and metastasis of tumors. In particular, overexpression or hyperactivity thereof in lung cancer, colorectal cancer, and breast cancer has been reported to contribute to inducing tumor invasion and metastasis and inhibiting apoptosis (Faham N. et. al., Cold Spring Harb Symp Quant Biol., 2016).

Accordingly, for effective cancer treatment, there is a need for a new anticancer agent applicable even to RON mutation-containing cancer that is resistant to conventional anticancer agents.

### DISCLOSURE OF INVENTION

### Technical Problem

An object of the present invention is to provide a pharmaceutical composition for preventing or treating cholangiocarcinoma, the pharmaceutical composition including, as an active ingredient, a compound capable of preventing or treating RON mutation-containing cholangiocarcinoma or a pharmaceutically acceptable salt thereof, and a method for preventing or treating cholangiocarcinoma using the pharmaceutical composition.

### Solution to Problem

To achieve the above-mentioned object, in an aspect of the present invention, there is provided a pharmaceutical composition for preventing or treating cholangiocarcinoma, the pharmaceutical composition including, as an active ingredient, a compound represented by Formula 1 or Formula 2, or a pharmaceutically acceptable salt thereof:

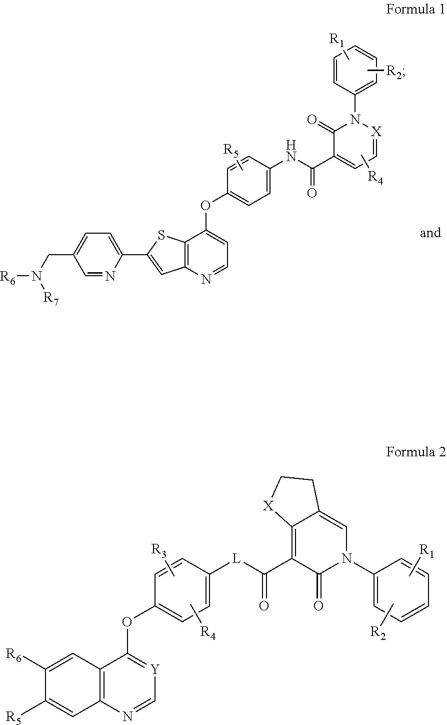

In another aspect of the present invention, there is provided a method for preventing or treating cholangiocarcinoma, the method including the steps of: detecting RON mutation in a biological sample derived from an individual suffering from cholangiocarcinoma, wherein the RON mutation is RONΔ155 in which exons 5, 6, and 11 are deleted, RONΔ160 in which exons 5 and 6 are deleted, or RONΔ165 in which exon 11 is deleted; and administering the pharmaceutical composition of claim 1 to the individual in which the RON mutation has been detected.

In still another aspect of the present invention, there is provided a method for providing information on an anticancer therapeutic agent, the method including the steps of: detecting RON mutation in a biological sample derived from an individual suffering from cholangiocarcinoma, wherein the RON mutation is RONΔ155 in which exons 5, 6, and 11 are deleted, RONΔ160 in which exons 5 and 6 are deleted, or RONΔ165 in which exon 11 is deleted; and providing, to the individual in which the RON mutation has been detected, information that the pharmaceutical composition of claim 1 is suitable for preventing or treating cholangiocarcinoma.

In yet another aspect of the present invention, there is provided a use of the pharmaceutical composition for preventing or treating cholangiocarcinoma.

In yet still another aspect of the present invention, there is provided a use of the pharmaceutical composition for preparing a medicament for preventing or treating cholangiocarcinoma.

### Advantageous Effects of Invention

The pharmaceutical composition for preventing or treating cancer according to the present invention is applicable to cholangiocarcinoma patients having RON mutation. In particular, the pharmaceutical composition can be effectively used in the treatment of cholangiocarcinoma patients who are resistant to cetuximab that is conventionally used for cancer treatment, and have mutation in RONΔ155, RONΔ160, or RONΔ165.

### BRIEF DESCRIPTION OF DRAWINGS

FIG. 1 illustrates cell death rates obtained after subjecting, to treatment with Sc shRNA or RON shRNA, KKU-213 cell line that is a cholangiocarcinoma cell line of mutant RONΔ160 type.

FIG. 2 illustrates results obtained by treating three cholangiocarcinoma cell lines with compounds of Example 1 (WM-S1-030) to Example 5 and BMS-777607 (positive control 1), and then confirming the cell proliferation inhibition efficacy depending on the RON genotype.

FIG. 3 illustrates results obtained by administering, to a mouse model transplanted with KKU-213 cell line that is a cholangiocarcinoma cell line of mutant RONΔ160 type, 30 mpk or 50 mpk of the compound of Example 1 or 50 mpk of the compound of positive control 1, and then confirming the tumor growth rate.

FIG. 4 illustrates results obtained by administering, to a mouse model transplanted with KKU-213 cell line that is a cholangiocarcinoma cell line of mutant RONΔ160 type, 30 mpk or 50 mpk of the compound of Example 1 or 50 mpk of the compound of positive control 1, and then performing immunochemical staining of the tumor tissue.

FIG. 5 illustrates results obtained by administering, to a mouse model transplanted with Choi-CK cell line that is a cholangiocarcinoma cell line of mutant RONΔ165 type, 30 mpk or 50 mpk of the compound of Example 1 or 50 mpk of the compound of positive control 1, and then confirming the tumor growth rate.

FIG. 6 illustrates results obtained by administering, to a mouse model transplanted with Choi-CK cell line that is a cholangiocarcinoma cell line of mutant RONΔ160 type, 30 mpk or 50 mpk of the compound of Example 1 or 50 mpk of the compound of positive control 1, and then the performing immunochemical staining of the tumor tissue.

FIG. 7 illustrates results obtained by administering, to a mouse model transplanted with CTG-0927 that is cancer tissue derived from a patient with cholangiocarcinoma of mutant RON Δ165 type, 10 mpk or 30 mpk of the compound of Example 1, and then confirming the tumor growth rate.

FIG. 8 illustrates results obtained by analyzing the mutated RON sequence in 14 cholangiocarcinoma cell lines.

FIG. 9 illustrates results obtained by analyzing the mutated RON sequence in 125 tissues from cholangiocarcinoma patients.

### BEST MODE FOR CARRYING OUT THE INVENTION

Hereinafter, the present invention will be described in more detail.

In an aspect of the present invention, there is provided a pharmaceutical composition for preventing or treating cholangiocarcinoma, the pharmaceutical composition including, as an active ingredient, a compound represented by Formula 1 or Formula 2, or a pharmaceutically acceptable salt thereof.

Here, the cholangiocarcinoma may have RON (Recepteur d'origine nantais) mutation. In particular, the cholangiocarcinoma may be resistant to EGFR-targeted therapeutic agents. In addition, the EGFR-targeted therapeutic agent may be at least one selected from the group consisting of cetuximab, gefitinib, erlotinib, apatinib, icotinib, brigatinib, lapatinib, canertinib, AEE788, XL647, zactima, and panitumumab.

In the present specification, the term “RON” refers to a protein encoded by human macrophage stimulating 1 receptor (MST1R) gene. RON is a protein receptor belonging to the c-MET family and is a receptor for macrophage-stimulating protein (MSP) that is a serum protein, is secreted by the liver, and regulates the action of macrophages. Ligand binding at the cell surface induces phosphorylation of RON in the intracellular domain, which provides a docking site for downstream signaling molecules. Signals from RON promote epithelial cell migration, proliferation, and survival at the wound site, thereby activating wound healing responses. RON regulates migration and phagocytic activity of macrophages, and thus plays a role in the innate immune response. In addition, RON can also promote signals such as cell migration and proliferation in response to growth factors other than MST1 ligand. RON is mainly expressed in epithelial cells of liver, lung, intestine, kidney, brain, bone, adrenal gland, skin, and the like.

The mutated form of RON is found in various solid cancers such as non-small cell lung cancer (NSCLC), small cell lung cancer (SCLC), pancreatic cancer, colorectal cancer, and cholangiocarcinoma. Here, the RON may have the nucleotide sequence of SEQ ID NOs: 1, 2, or 3.

As used herein, the term “MST1R gene” refers to a tyrosine kinase receptor that binds to MST1 ligand, thereby transmitting a signal to the cytoplasm. This gene regulates a variety of physiological processes including cell survival, migration, and differentiation.

As used herein, the term “RON mutation” may be characterized by being RONΔ155 where exons 5, 6, and 11 are deleted, RONΔ160 where exons 5 and 6 are deleted, or RONΔ165 where exon 11 is deleted. The cDNA of RONΔ155 may have the nucleotide sequence of SEQ ID NO: 1, the cDNA of RONΔ160 may have the nucleotide sequence of SEQ ID NO: 2, and the cDNA of RONΔ165 may have the nucleotide sequence of SEQ ID NO: 3.

As used herein, the term “resistance” means that no efficacy is observed for a drug because sensitive responses to the drug do not occur.

As used herein, the term “EGFR-targeted therapeutic agent” refers to an anticancer agent that targets EGFR. Any EGFR-targeted therapeutic agent may be applied as long as it exhibits an anticancer effect. The EGFR-targeted therapeutic agent may be preferably cetuximab, gefitinib, erlotinib, apatinib, icotinib, brigatinib, lapatinib, canertinib, AEE788, XL647, zactima or panitumumab, most preferably, cetuximab.

The compound of Formula 1 used in the present invention is represented as below:

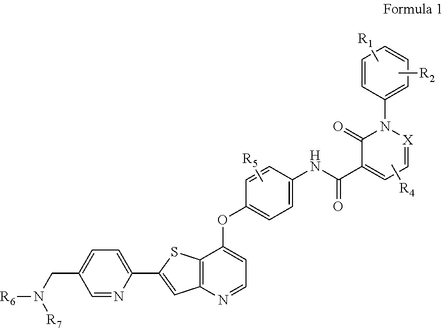

In Formula 1 above,
R₁ and R₂ are each independently H, halogen, C₁₋₁₀ alkoxy, or halo C₁₋₁₀alkyl;
X is —C(—R₃)═ or —N═;
R₃ and R₄ are each independently H, halogen, C₁₋₁₀ alkyl, or C₁₋₁₀ alkoxy;
R₅ is H, halogen, or C₁₋₁₀alkyl;
R₆ and R₇ form a 4- to 10-membered heterocycle together with the N atom to which they are bonded, or R₆ is —C₂H₄O—CH₃, and R₇ is H, methyl, or t-butoxycarbonyl; and
the heterocycle optionally further has one or two heteroatoms selected from the group consisting of N, O, and S, in addition to the N atom to which R₆ and R₇ are bonded, and is unsubstituted or substituted with one or more substituents selected from among halogen and C₁₋₆ alkyl,
the C₁₋₁₀ alkyl may include C₁₋₆ alkyl, C₁₋₃ alkyl, C₃₋₁₀ alkyl, C₃₋₆ alkyl, C₆₋₁₀ alkyl, and the like. In addition, the C₁₋₁₀ alkoxy may include C₁₋₆ alkoxy, C₁₋₃ alkoxy, C₃₋₁₀ alkoxy, C₃₋₆ alkoxy, C₆₋₁₀ alkoxy, and the like. In addition, the 4- to 10-membered heterocycle may include 4- to 7-membered heterocycle, 4- to 6-membered heterocycle, 5- to 7-membered heterocycle, 5- or 6-membered heterocycle, and the like.

According to one embodiment, in Formula 1 above, R₁ and R₂ may be each independently H, halogen, methoxy, or —CF₃. Here, the halogen may be F, Cl, Br, or I.

According to another embodiment, in Formula 1 above, R₃ and R₄ may be each independently H, halogen, methyl, methoxy, or ethoxy. Here, the halogen may be F, Cl, Br, or I.

According to a further embodiment, in Formula 1 above, X is —C(—R₃)═; and R₃ and R₄ are each independently H, halogen, methyl, methoxy, or ethoxy, but are not simultaneously H.

According to a still further embodiment, in Formula 1 above, X may be —N═; and R₄ may be halogen, methyl, methoxy, or ethoxy, Here, the halogen may be F, Cl, Br, or I.

According to a still further embodiment, in Formula 1 above, R₅ may be H or halogen, Here, the halogen may be F, Cl, Br, or I.

According to a still further embodiment, in Formula 1 above, R₆ and R₇, taken together with the N atom to which they are bonded, form

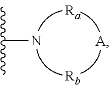

wherein Rₐ and R_{b} may be each independently C₁₋₃ alkylene, A may be —N(—R₉)— or —O—, and R₉ may be C₁₋₆ alkyl. As specific examples, R₆ and R₇, together with the N atom to which they are bonded, may form a heterocycle group such as azetidinyl, diazetidinyl, pyrrolidinyl, pyrrolyl, imidazolidinyl, imidazolyl, pyrazolidinyl, pyrazolyl, oxazolidinyl, oxazolyl, isoxazolidinyl, isoxazolyl, thiazolidinyl, thiazolyl, isothiazolidinyl, isothiazolyl, piperidinyl, pyridinyl, piperazinyl, diazinyl, morpholino, thiomorpholino, azepanyl, and diazepanyl, which is optionally substituted with C₁₋₆ alkyl. Further, Rₐ and R_{b} may be each independently —CH₂—, —C₂H₄—, or —C₃H₆—. In addition, R₉ may be methyl, ethyl, n-propyl, i-propyl, n-butyl, i-butyl, t-butyl, n-pentyl, i-pentyl, t-pentyl, sec-pentyl, neopentyl, hexyl, and the like.

According to a still yet further embodiment, in Formula 1 above, R₁ and R₂ are each independently H, halogen, methoxy, or —CF₃; R₃ and R₄ may be each independently H, halogen, methyl, methoxy, or ethoxy; R₅ may be H or halogen; and R₆ may be —C₂H₄O—CH₃ and R₇ may be H, methyl, or t-butoxycarbonyl, or R₆ and R₇ may be bonded together to form morpholino or methylpiperazinyl. Here, the halogen may be F, Cl, Br, or I.

According to one embodiment, the compound of Formula 1 above may be represented by Formula 1a below:

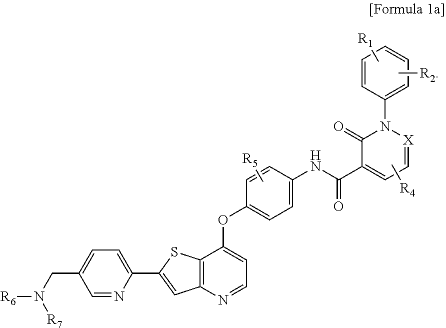

In Formula 1a above, R₁ to R₇ are the same as defined in Formula 1 above.

Specifically, in Formula 1a above, R₁ and R₂ may be each independently H, halogen, or —CF₃. Further, R₃ and R₄ may be each independently H, halogen, methyl, methoxy, or ethoxy, but are not simultaneously H. In addition, R₅ may be H or halogen.

More specifically, in Formula 1a above, R₁ and R₂ may be each independently H, halogen, or —CF₃; R₃ and R₄ may be each independently H, halogen, methyl, methoxy, or ethoxy; R₅ may be H or halogen; R₆ may be —C₂H₄O—CH₃; and R₇ may be H, methyl, or t-butoxycarbonyl.

According to another embodiment, the compound of Formula 1 above may be represented by Formula 1b below:

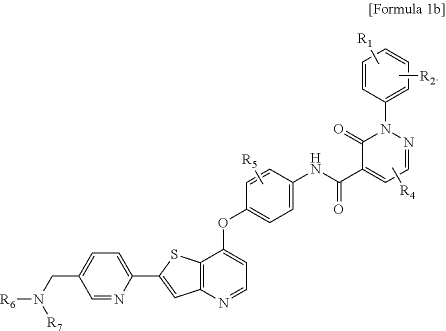

In Formula 1b above, R₁ to R₇ are the same as defined in Formula 1 above.

Specifically, in Formula 1b above, R₁ and R₂ may be each independently H, halogen, or —CF₃. Further, R₄ may be halogen, methyl, methoxy, or ethoxy. In addition, R₅ may be H or halogen.

More specifically, in Formula 1b above, R₁ and R₂ may be each independently H, halogen, or —CF₃; R₄ may be halogen, methyl, methoxy, or ethoxy; R₅ may be H or halogen; R₆ may be —C₂H₄O—CH₃; and R₇ may be H, methyl, or t-butoxycarbonyl.

According to a further embodiment, the compound of Formula 1 above may be represented by Formula 1c below:

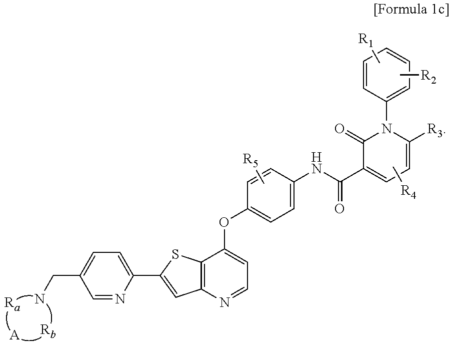

In Formula 1c above R₁ to R₅ may be the same as defined in Formula 1 above; Rₐ and R_{b} may be each independently C₁₋₃ alkylene; A may be —N(—R₉)— or —O—; and R₉ may be C₁₋₆ alkyl.

Specifically, in Formula 1c above, R₁ and R₂ may be each independently H, halogen, or —CF₃. Further, R₃ and R₄ may be each independently H, halogen, methyl, methoxy, or ethoxy, but are not simultaneously H. In addition, R₅ may be H or halogen. Further, Rₐ and R_{b} may form morpholino or methylpiperazinyl together with N and A to which they are bonded.

According to a further embodiment, the compound of Formula 1 may be represented by Formula 1d below.

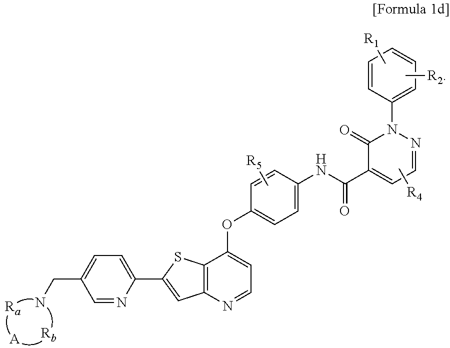

In Formula 1d above, R₁ to R₅ may be the same as defined in Formula 1 above; Rₐ and R_{b} may be each independently C₁₋₃ alkylene; A may be —N(—R₉)— or —O—; and R₉ may be C₁₋₆ alkyl.

Specifically, in Formula 1d above, R₁ and R₂ may be each independently H, halogen, or —CF₃. Further, R₄ may be halogen, methyl, methoxy, or ethoxy. In addition, R₅ may be H or halogen. Further, Rₐ and R_{b} may form morpholino or methylpiperazinyl together with N and A to which they are bonded.

Specific examples of the compound of Formula 1 are listed below:
1) 4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1-phenyl-1,2-dihydropyridine-3-carboxamide;
2) N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-4-methoxy-2-oxo-1,2-dihydropyridine-3-carboxamide;
3) N-(3-fluoro-4-{[2-(5-{[(2-methoxyethyl)amino]methyl}pyridin-2-yl)thieno[3,2-b]pyridin-7-yl]oxy}phenyl)-4-methoxy-2-oxo-1-phenyl-1,2-dihydropyridine-3-carboxamide;
4) N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-2-oxo-1,2-dihydropyridine-3-carboxamide;
5) N-(3-fluoro-4-{[2-(5-{[(2-methoxyethyl)amino]methyl}pyridin-2-yl)thieno[3,2-b]pyridin-7-yl]oxy}phenyl)-2-oxo-1-phenyl-1,2-dihydropyridine-3-carboxamide;
6) t-butyl {[6-(7-{4-[4-ethoxy-1-(4-fluorophenyl)-2-oxo-1,2-dihydropyridine-3-carboxamido]-2-fluorophenoxy}thieno[3,2-b]pyridin-2-yl)pyridin-3-yl]methyl}(2-methoxyethyl)carbamate;
7) 4-ethoxy-N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-2-oxo-1,2-dihydropyridine-3-carboxamide;
8) 1-(4-chlorophenyl)-4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1,2-dihydropyridine-3-carboxamide;
9) N-(3-chloro-4-{[2-(5-{[(2-methoxyethyl)amino]methyl}pyridin-2-yl)thieno[3,2-b]pyridin-7-yl]oxy}phenyl)-1-(4-fluorophenyl)-4-methoxy-2-oxo-1,2-dihydropyridine-3-carboxamide;
10) N-(2-chloro-4-{[-2-(5-{[(2-methoxyethyl)amino]methyl}pyridin-2-yl)thieno[3,2-b]pyridin-7-yl]oxy}phenyl)-1-(4-fluorophenyl)-4-methoxy-2-oxo-1,2-dihydropyridine-3-carboxamide;
11) 1-(4-fluorophenyl)-4-methoxy-N-(-4-{[2-(5-{[(2-methoxyethyl)amino]methyl}pyridin-2-yl)thieno[3,2-b]pyridin-7-yl)oxy)phenyl)-2-oxo-1,2-dihydropyridine-3-carboxamide;
12) 4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1-(4-(trifluoromethyl)phenyl)-1,2-dihydropyridine-3-carboxamide;
13) 1-(4-chlorophenyl)-N-(3-fluoro-4-{[2-(5-{[(2-methoxyethyl)amino]methyl}pyridin-2-yl)thieno[3,2-b]pyridin-7-yl]oxy}phenyl)-4-methoxy-2-oxo-1,2-dihydropyridine-3-carboxamide;
14) 4-ethoxy-N-(3-fluoro-4-{[2-(5-{[(2-methoxyethyl)amino]methyl}pyridin-2-yl)thieno[3,2-b]pyridin-7-yl]oxy}phenyl)-1-(3-fluorophenyl)-2-oxo-1,2-dihydropyridine-3-carboxamide;
15) 4-ethoxy-N-(3-fluoro-4-{[2-(5-{[(2-methoxyethyl)amino]methyl}pyridin-2-yl)thieno[3,2-b]pyridin-7-yl]oxy}phenyl)-1-(4-methoxyphenyl)-2-oxo-1,2-dihydropyridine-3-carboxamide;
16) 4-ethoxy-N-(3-fluoro-4-{[2-(5-{[(2-methoxyethyl)amino]methyl}pyridin-2-yl)thieno[3,2-b]pyridin-7-yl]oxy}phenyl)-1-(3-methoxyphenyl)-2-oxo-1,2-dihydropyridine-3-carboxamide;
17) N-(3-fluoro-4-{[2-(5-{[(2-methoxyethyl)amino]methyl}pyridin-2-yl)thieno[3,2-b]pyridin-7-yl]oxy}phenyl)-2-(4-fluorophenyl)-5-methyl-3-oxo-2,3-dihydropyridazine-4-carboxamide;
18) N-(3-fluoro-4-{[2-(5-{[(2-methoxyethyl)amino]methyl}pyridin-2-yl)thieno[3,2-b]pyridin-7-yl]oxy}phenyl)-5-methyl-3-oxo-2-phenyl-2,3-dihydropyridazine-4-carboxamide;
19) N-(3-fluoro-4-{[2-(5-{[(2-methoxyethyl)amino]methyl}pyridin-2-yl)thieno[3,2-b]pyridin-7-yl]oxy}phenyl)-3-oxo-2-phenyl-2,3-dihydropyridazine-4-carboxamide;
20) N-(3-fluoro-4-[{2-(5-[{(2-methoxyethyl)amino]methyl}pyridin-2-yl)thieno[3,2-b]pyridin-7-yl}oxy]phenyl)-2-(4-fluorophenyl)-3-oxo-2,3-dihydropyridazine-4-carboxamide;
21) N-(3-fluoro-4-{[2-(5-{[(2-methoxyethyl)amino]methyl}pyridin-2-yl)thieno[3,2-b]pyridin-7-yl]oxy}phenyl)-6-methyl-2-oxo-1-phenyl-1,2-dihydropyridine-3-carboxamide;
22) N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-6-methyl-2-oxo-1,2-dihydropyridine-3-carboxamide;
23) 5-bromo-N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-2-oxo-1,2-dihydropyridine-3-carboxamide;
24) 5-chloro-N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-2-oxo-1,2-dihydropyridine-3-carboxamide;
25) N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-4-methyl-2-oxo-1,2-dihydropyridine-3-carboxamide;
26) N-(2-chloro-4-{[2-(5-{[(2-methoxyethyl)amino]methyl}pyridin-2-yl)thieno[3,2-b]pyridin-7-yl]oxy}phenyl)-4-ethoxy-1-(4-fluorophenyl)-2-oxo-1,2-dihydropyridine-3-carboxamide;
27) N-(3-fluoro-4-([2-(5-{[(2-methoxyethyl)amino)methyl]pyridin-2-yl}thieno[3,2-b]pyridin-7-yl)oxy]phenyl}-1-(4-fluorophenyl)-5,6-dimethyl-2-oxo-1,2-dihydropyridine-3-carboxamide;
28) N-(3-fluoro-4-{[2-(5-{[(2-methoxyethyl)amino]methyl}pyridin-2-yl)thieno[3,2-b]pyridin-7-yl]oxy}phenyl)-4-methyl-2-oxo-1-phenyl-1,2-dihydropyridine-3-carboxamide;
29) N-(3-fluoro-4-{[2-(5-{[(2-methoxyethyl)amino]methyl}pyridin-2-yl)thieno[3,2-b]pyridin-7-yl]oxy}phenyl)-1-(4-fluorophenyl)-5-methyl-2-oxo-1,2-dihydropyridine-3-carboxamide;
30) 4-ethoxy-N-(3-fluoro-4-{[2-(5-{[(2-methoxyethyl)(methyl)amino]methyl}pyridin-2-yl)thieno[3,2-b]pyridin-7-yl]oxy}phenyl)-1-(4-fluorophenyl)-2-oxo-1,2-dihydropyridine-3-carboxamide;
31) 4-ethoxy-N-(3-fluoro-4-({2-[5-(morpholinomethyl)pyridin-2-yl]thieno[3,2-b]pyridin-7-yl}oxy)phenyl]-1-(4-fluorophenyl)-2-oxo-1,2-dihydropyridine-3-carboxamide;
32) 4-ethoxy-N-[3-fluoro-4-({2-(5-(morpholinomethyl)pyridin-2-yl]thieno[3,2-b]pyridin-7-yl]oxy}phenyl)-2-oxo-1-phenyl-1,2-dihydropyridine-3-carboxamide;
33) 4-ethoxy-N-{3-fluoro-4-[(2-{5-[(4-methylpiperazin-1-yl)methyl]pyridin-2-yl}thieno[3,2-b]pyridin-7-yl)oxy]phenyl}-1-(4-fluorophenyl)-2-oxo-1,2-dihydropyridine-3-carboxamide;
34) 4-ethoxy-N-{3-fluoro-4-[(2-{5-[(methylpiperazin-1-yl)methyl]pyridin-2-yl}thieno[3,2-b]pyridin-7-yl)oxy]phenyl}-2-oxo-1-phenyl-1,2-dihydropyridine-3-carboxamide;
35) 1-(4-chlorophenyl)-4-ethoxy-N-[3-fluoro-4-{[2-[5-(morpholinomethyl)pyridin-2-yl]thieno[3,2-b]pyridin-7-yl}oxy)phenyl]-2-oxo-1,2-dihydropyridine-3-carboxamide;
36) N-(3-chloro-4-({2-[5-(morpholinomethyl)pyridin-2-yl]thieno[3,2-b]pyridin-7-yl}oxy)phenyl]-4-ethoxy-1-(4-fluorophenyl)-2-oxo-1,2-dihydropyridine-3-carboxamide; and
37) N-[2-chloro-4-{[2-[5-(morpholinomethyl)pyridin-2-yl]thieno[3,2-b]pyridin-7-yl}oxy)phenyl]-4-ethoxy-1-(4-fluorophenyl)-2-oxo-1,2-dihydropyridine-3-carboxamide.

The compound represented by Formula 1 above may be a compound selected from the group consisting of:
4-ethoxy-N-[3-fluoro-4-({2-[5-(morpholinomethyl)pyridin-2-yl]thieno[3,2-b]pyridin-7-yl}oxy)phenyl]-1-(4-fluorophenyl)-2-oxo-1,2-dihydropyridine-3-carboxamide;
4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1-phenyl-1,2-dihydropyridine-3-carboxamide; and
4-ethoxy-N-(3-fluoro-4-[(2-{5-[(methylpiperazin-1-yl)methyl]pyridin-2-yl}thieno[3,2-b]pyridin-7-yl)oxy]phenyl}-2-oxo-1-phenyl-1,2-dihydropyridine-3-carboxamide.

The compound of Formula 1 above used in the composition according to the present invention may be prepared by a method disclosed in Korean Patent No. 10-2221689, and by other known methods and/or various methods based on the technology in the field of organic synthesis. Based on the above methods, various derivatives may be synthesized using an appropriate synthesis method according to the type of substituent.

The compound of Formula 2 used in the present invention is represented as below:

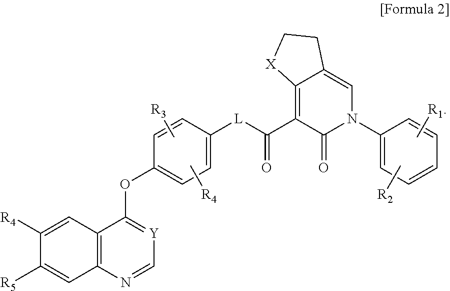

In Formula 2 above,
L is —NH— or —CH₂—,
R₁ to R₄ are each independently hydrogen, halogen, hydroxy, cyano, C₁₋₄ alkyl, C₁₋₄ alkoxy, C₁₋₄ haloalkyl, C₂₋₄ alkenyl, C₂₋₄ alkynyl, C₃₋₇ cycloalkyl, C₆₋₁₀ aryl, 5- to 9-membered heteroaryl, or 3- to 9-membered heterocycloalkyl,
X is O, S, —CH(-Rx)-, or —N(-Rx)-,
Rx is hydrogen, C₁₋₄ alkyl, C₁₋₄ alkoxy, C₁₋₄ haloalkyl, C₂₋₄ alkenyl, C₂₋₄ alkynyl, C₆₋₁₀ aryl, C₆₋₁₀ aryl-C₁₋₄ alkyl, or 3- to 9-membered heterocycloalkyl,
Y is —N═ or —CH═, and
R₅ and R₆ are each independently hydrogen, amino, halogen, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylcarbonylamino, C₁₋₆ alkylcarbonylamino, C₁₋₆ alkylamino, or C₁₋₆ alkyl-amino-C₁₋₆ alkoxy,
wherein R₅ and R₆ are each independently unsubstituted or substituted with 3- to 9-membered cycloalkyl or 3- to 9-membered heterocycloalkyl,
the cycloalkyl or the heterocycloalkyl optionally has one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl, and
the heterocycloalkyl contains 1 to 4 heteroatoms selected from the group consisting of N, O, and S.

The C₁₋₆ alkyl may include C₁₋₃ alkyl, C₃₋₆ alkyl, and the like. In addition, the C₁₋₆ alkoxy may include C₁₋₃ alkoxy, C₃₋₆ alkoxy, and the like.

According to an embodiment, in Formula 2 above, R₁ to R₄ may be each independently hydrogen, C₁₋₄ haloalkyl, or halogen. Here, the halogen may be F, Cl, Br, or I. Specifically, R₁ may be hydrogen, trifluoromethyl, or fluoro; R₂ may be hydrogen; R₃ may be fluoro; and R₄ may be hydrogen.

According to another embodiment, in Formula 2 above, X may be O or —CH(-Rx)- and Rx may be hydrogen or C₁₋₆ alkyl.

According to still yet another embodiment, in Formula 2 above, R₅ and R₆ may be each independently hydrogen, amino, halogen, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylcarbonylamino, C₁₋₆ alkylcarbonylamino, C₁₋₆ alkylamino, C₁₋₆ alkyl-amino-C₁₋₆ alkoxy, or 5- to 9-membered heteroaryl, wherein R₅ and R₆ may be each independently unsubstituted or substituted with: C₁₋₆ alkyl; C₁₋₆ alkyl or C₁₋₆ alkyl-amino-C₁₋₆ alkyl, which is substituted with any one among C₁₋₆ alkoxy-C₁₋₆ alkylamino, 3- to 9-membered cycloalkyl, and 3- to 9-membered heterocycloalkyl; 3- to 9-membered cycloalkyl; or 3- to 9-membered heterocycloalkyl; the cycloalkyl or the heterocycloalkyl may optionally have one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl, and the heteroaryl and the heterocycloalkyl may each independently contain one or more heteroatoms selected from the group consisting of N, O, and S.

Specifically, the heteroaryl may be pyridinyl, imidazolyl, or pyrazolyl; the heterocycloalkyl may be azetidinyl, pyrrolidinyl, tetrahydropyranyl, morpholino, morpholinyl, dioxidothiomorpholino, piperazinyl, piperidinyl, or oxetanyl, and the cycloalkyl may be cyclobutyl, cyclopentyl, or cyclohexyl.

In addition, in a case where the heteroaryl or the heterocycloalkyl contains one or more N atoms, substitution may be made at any one of the N atomic positions. However, there is no particular limitation thereon.

According to still yet another embodiment, in Formula 2 above, R₁ and R₂ may be hydrogen, C₁₋₄ haloalkyl, or halogen; R₃ and R₄ may be hydrogen or halogen; X may be O or —CH(-Rx)- and Rx may be hydrogen or C₁₋₄ alkyl; A may be quinoline, quinazoline, pyridine, pyrimidine, thienopyridine, pyrrolopyridine, pyrazolopyridine, imidazopyridine, pyrrolopyrimidine, dihydropyrrolopyrimidine, furopyridine, pyrazolopyrimidine, purine, or indazole; and R₅ and R₆ may be each independently hydrogen, amino, halogen, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylcarbonylamino, C₁₋₆ alkylcarbonylamino, C₁₋₆ alkylamino, C₁₋₆ alkyl-amino-C₁₋₆ alkoxy, or 5- to 9-membered heteroaryl, wherein R₅ and R₆ may be each independently unsubstituted or substituted with: C₁₋₆ alkyl; C₁₋₆ alkyl or C₁₋₆ alkyl-amino-C₁₋₆ alkyl, which is substituted with any one of C₁₋₆ alkoxy-C₁₋₆ alkyl-amino, 3- to 9-membered cycloalkyl, and 3- to 9-membered heterocycloalkyl; 3- to 9-membered cycloalkyl; or 3- to 9-membered heterocycloalkyl; the cycloalkyl or the heterocycloalkyl may optionally have one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl; and the heteroaryl and the heterocycloalkyl may each independently contain one or more heteroatoms selected from the group consisting of N, O, and S.

According to still yet another embodiment, in Formula 2 above, R₅ and R₆ are each independently hydrogen, nitro, amino, halogen, hydroxy, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylaminocarbonyl, C₁₋₆ alkylcarbonylamino, C₁₋₆ alkylamino, C₁₋₆ alkyl-amino-C₁₋₆ alkoxy, C₆₋₁₀ aryl, C₆₋₁₀ aryl-C₁₋₄ alkyl, or 5- to 9-membered heteroaryl, wherein the amino, the alkyl, the alkoxy, the aryl, and the heteroaryl may be each independently unsubstituted or substituted with: C₁₋₆ alkyl; C₁₋₆ alkyl or C₁₋₆ alkyl-amino-C₁₋₆ alkyl, which is substituted with any one of C₁₋₆ alkoxy-C₁₋₆ alkyl-amino, 3- to 9-membered cycloalkyl, and 3- to 9-membered heterocycloalkyl; 3- to 9-membered cycloalkyl; or 3- to 9-membered heterocycloalkyl; the cycloalkyl or the heterocycloalkyl may optionally have one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl; and the heterocyclic ring, the heteroaryl and the heterocycloalkyl may each independently contain one or more heteroatoms selected from the group consisting of N, O, and S.

According to still yet further another embodiment, in Formula 2 above, R₅ and R₆ may not be, at the same time, C₆₋₁₀ aryl, C₆₋₁₀ aryl-C₁₋₄ alkyl, or 5- to 9-membered heteroaryl. According to still yet another embodiment, in Formula 1 above, R₅ and R₆ may not be substituted, at the same time, with C₁₋₆ alkyl or C₁₋₆ alkylamino-C₁₋₆ alkyl, which is substituted with any one of 3- to 9-membered cycloalkyl and 3- to 9-membered heterocycloalkyl; 3- to 9-membered cycloalkyl; or 3- to 9-membered heterocycloalkyl. As a specific example, in a case where R₅ contains a ring such as aryl or heteroaryl, R₆ may not contain this ring at the same time. In addition, in a case where R₅ is substituted with a group containing a ring such as cycloalkyl or heterocycloalkyl, R₆ may not be substituted, at the same time, with a group containing this ring.

As a more specific example, R₅ may be C₆₋₁₀ aryl, C₆₋₁₀ aryl-C₁₋₄ alkyl, or 5- to 9-membered heteroaryl; and R₆ may be hydrogen, nitro, amino, halogen, hydroxy, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylaminocarbonyl, C₁₋₆ alkylcarbonylamino, C₁₋₆ alkylamino, or C₁₋₆ alkyl-amino-C₁₋₆ alkoxy. Here, R₅ may be unsubstituted or substituted with C₁₋₆ alkyl; or C₁₋₆ alkyl or C₁₋₆ alkylamino-C₁₋₆ alkyl, which is substituted with any one of C₁₋₆ alkoxy-C₁₋₆ alkylamino, 3- to 9-membered cycloalkyl, and 3- to 9-membered heterocycloalkyl. In addition, R₆ may be unsubstituted or substituted with 3- to 9-membered cycloalkyl or 3- to 9-membered heterocycloalkyl. Here, the cycloalkyl or the heterocycloalkyl may optionally have one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl; and the heteroaryl and the heterocycloalkyl may each independently contain one or more heteroatoms selected from the group consisting of N, O, and S.

According to still yet another embodiment, in Formula 2 above, R₅ and R₆ may be each independently hydrogen, amino, halogen, hydroxy, C₁₋₆ alkoxy, aminocarbonyl, C₁₋₆ alkylaminocarbonyl, diC₁₋₆ alkylaminocarbonyl, C₁₋₆ alkylcarbonylamino, cyano, C₁₋₄ haloalkyl, C₁₋₆ alkyl, 5- or 9-membered heteroaryl, Ax-(CH₂)ₐ-L1-(CH₂)_{b}-L2-, or Ax-(CH₂)ₐ-L1-(CH₂)_{b}-L2-pyridinyl; Ax is C₃₋₆ cycloalkyl or 3-6 membered heterocycloalkyl; L1 and L2 may be each independently a single bond, —O—, —NH—, —C(═O)—NH—, or —NH—C(═O)—; and a and b may be each independently an integer of 0 to 3, provided that in a case where b is 0, L2 is a single bond, wherein the cycloalkyl, the heteroaryl, and the heterocycloalkyl may each independently optionally have one or two substituents selected from the group consisting of halogen, oxo, cyano, hydroxy, hydroxymethyl, C₁₋₆ alkyl, methoxy, methoxymethyl, dimethylamino, and methoxyethylaminomethyl; the heteroaryl and the heterocycloalkyl may each independently contain one or more heteroatoms selected from the group consisting of N, O, and S; and the heteroaryl and the heterocycloalkyl may each independently contain one or more heteroatoms selected from the group consisting of N, O, and S.

Specific examples of the compound represented by Formula 2 above are as follows:
40) N-(4-((6,7-dimethoxyquinolin-4-yl)oxy)-3-fluorophenyl)-5-(4-fluorophenyl)-6-oxo-2,3,5,6-tetrahydrofuro[3,2-c]pyridine-7-carboxamide;
41) N-(4-((6,7-dimethoxyquinolin-4-yl)oxy)-3-fluorophenyl)-6-oxo-5-phenyl-2,3,5,6-tetrahydrofuro[3,2-c]pyridine-7-carboxamide;
42) N-(4-((6,7-dimethoxyquinolin-4-yl)oxy)-3-fluorophenyl)-6-oxo-5-(4-trifluoromethyl)phenyl)-2,3,5,6-tetrahydrofuro[3,2-c]pyridine-7-carboxamide;
43) N-(4-((6,7-dimethoxyquinolin-4-yl)oxy)-3-fluorophenyl)-6-oxo-5-(3-fluorophenyl)-2,3,5,6-tetrahydrofuro[3,2-c]pyridine-7-carboxamide;
44) N-(3-fluoro-4-((7-methoxyquinolin-4-yl)oxy)phenyl)-5-(4-fluorophenyl)-6-oxo-2,3,5,6-tetrahydrofuro[3,2-c]pyridine-7-carboxamide;
45) N-(3-fluoro-4-((7-methoxyquinolin-4-yl)oxy)phenyl)-5-(4-fluorophenyl)-6-oxo-2,3,5,6-tetrahydrofuro[3,2-c]pyridine-7-carboxamide;
46) N-(4-((6,7-dimethoxyquinazolin-4-yl)oxy)-3-fluorophenyl)-5-(4-fluorophenyl)-6-oxo-2,3,5,6-tetrahydrofuro[3,2-c]pyridine-7-carboxamide;
47) N-(4-((6-carbamoyl-7-methoxyquinolin-4-yl)oxy)-3-fluorophenyl)-5-(4-fluorophenyl)-6-oxo-2,3,5,6-tetrahydrofuro[3,2-c]pyridine-7-carboxamide;
48) N-(3-fluoro-4-((7-methoxy-6-(methylcarbamoyl)quinolin-4-yl)oxy)phenyl)-5-(4-fluorophenyl)-6-oxo-2,3,5,6-tetrahydrofuro[3,2-c]pyridine-7-carboxamide;
49) N-(4-((6-(dimethylcarbamoyl)-7-methoxyquinolin-4-yl)oxy)-3-fluorophenyl)-5-(4-fluorophenyl)-6-oxo-2,3,5,6-tetrahydrofuro[3,2-c]pyridine-7-carboxamide;
50) N-(3-fluoro-4-((7-methoxy-6-((2-morpholinoethyl)carbamoyl)quinolin-4-yl)oxy)phenyl)-5-(4-fluorophenyl)-6-oxo-2,3,5,6-tetrahydrofuro[3,2-c]pyridine-7-carboxamide;
51) N-(4-((6-(ethylcarbamoyl)-7-methoxyquinolin-4-yl)oxy)-3-fluorophenyl)-5-(4-fluorophenyl)-6-oxo-2,3,5,6-tetrahydrofuro[3,2-c]pyridine-7-carboxamide;
52) N-(4-((6-acetamido-7-methoxyquinolin-4-yl)oxy)-3-fluorophenyl)-5-(4-fluorophenyl)-6-oxo-2,3,5,6-tetrahydrofuro[3,2-c]pyridine-7-carboxamide;
53) N-(3-fluoro-4-((7-methoxy-6-(2-morpholinoacetamido)quinolin-4-yl)oxy)phenyl)-5-(4-fluorophenyl)-6-oxo-2,3,5,6-tetrahydrofuro[3,2-c]pyridine-7-carboxamide;
76) N-(3-fluoro-4-((6-methoxy-7-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
77) N-(3-fluoro-4-((6-methoxy-7-(3-morpholinopropoxy)quinolin-4-yl)oxy)phenyl)-2-(4-fluorophenyl)-3-oxo-3,5,6,7-tetrahydrofuro-2H-cyclopenta[c]pyridine-4-carboxamide;
78) N-(3-fluoro-4-((6-methoxy-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
79) N-(3-fluoro-4-((6-methoxy-7-(2-morpholinoethoxy)quinolin-4-yl)oxy)phenyl)-6-oxo-5-phenyl-2,3,5,6-tetrahydrofuro[3,2-c]pyridine-7-carboxamide;
80) N-(3-fluoro-4-((6-methoxy-7-(3-(4-methylpiperazin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
81) N-(3-fluoro-4-((7-(3-(3-hydroxyazetidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
82) N-(3-fluoro-4-((7-(3-(3-hydroxy-3-methylazetidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
83) N-(3-fluoro-4-((7-(3-(hydroxymethyl)azetidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
84) N-(3-fluoro-4-((6-methoxy-7-(3-(3-methoxyazetidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
85) N-(3-fluoro-4-((6-methoxy-7-(3-(3-methoxy-3-methylazetidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
86) N-(3-fluoro-4-((7-(3-(3-fluoroazetidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
87) N-(4-((7-(3-(3-(3,3-difluoroazetidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
88) N-(4-((7-(3-(3-cyanoazetidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
89) N-(3-fluoro-4-((6-methoxy-7-(3-(3-methylazetidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
90) N-(3-fluoro-4-((7-(3-(3-hydroxypyrrolidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
91) N-(3-fluoro-4-((7-(3-(3-hydroxy-3-methylpyrrolidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
92) N-(3-fluoro-4-((6-methoxy-7-(3-(3-methoxypyrrolidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
93) N-(3-fluoro-4-((6-methoxy-7-(3-(pyrrolidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
94) N-(3-fluoro-4-((7-(3-(3-fluoropyrrolidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
95) N-(4-((7-(3-(3-(3,3-difluoropyrrolidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
96) N-(3-fluoro-4-((7-(3-(4-hydroxypiperidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
97) N-(3-fluoro-4-((7-(3-(3-hydroxypiperidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
98) N-(3-fluoro-4-((7-(3-(4-hydroxy-4-methylpiperidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
99) N-(3-fluoro-4-(6-methoxy-7-(3-(4-methoxypiperidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
100) N-(3-fluoro-4-((6-methoxy-7-(3-(3-methoxypiperidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
101) N-(3-fluoro-4-((6-methoxy-7-(3-(4-oxopiperidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
102) N-(4-((7-(3-(1,1-dioxydothiomorpholino)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
103) N-(3-fluoro-4-(6-methoxy-7-(3-(piperidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
104) N-(3-fluoro-4-((7-(3-(4-fluoropiperidin-1-yl)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
105) N-(4-((7-(3-(4,4-difluoropiperidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
106) N-(3-fluoro-4-((6-methoxy-7-(3-(4-methylpiperidin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
107) N-(4-((7-(3-(3-(4,4-dimethylpiperidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
108) N-(3-fluoro-4-((7-(3-((3-hydroxycyclobutyl)amino)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
109) N-(3-fluoro-4-((6-methoxy-7-(3-((3-methoxycyclobutyl)amino)propoxy)quinolin-4-yl)oxy)phenyl)-5-(4-fluorophenyl)-6-oxo-2,3,5,6-tetrahydrofuro[3,2-c]pyridine-7-carboxamide;
110) N-(3-fluoro-4-((6-methoxy-7-(3-(oxetan-3-ylamino)propoxy)quinolin-4-yl)oxy)phenyl)-5-(4-fluorophenyl)-6-oxo-2,3,5,6-tetrahydrofuro[3,2-c]pyridine-7-carboxamide;
111) N-(3-fluoro-4-((6-methoxy-7-(3-((oxetan-3-ylmethyl)amino)propoxy)quinolin-4-yl)oxy)phenyl)-5-(4-fluorophenyl)-6-oxo-2,3,5,6-tetrahydrofuro[3,2-c]pyridine-7-carboxamide;
112) N-(3-fluoro-4-((7-(3-((3-hydroxycyclopentyl)amino)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
113) N-(3-fluoro-4-((7-(3-((3-hydroxycyclohexyl)amino)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
114) N-(3-fluoro-4-((7-(3-(((3-hydroxycyclohexyl)amino)propoxy)-6-methoxyquinolin-4-yl)oxy)phenyl)-5-(4-fluorophenyl)-6-oxo-2,3,5,6-tetrahydrofuro[3,2-c]pyridine-7-carboxamide;
115) N-(3-fluoro-4-((6-methoxy-7-(3-((tetrahydro-2H-pyran-4-yl)amino)propoxy)quinolin-4-yl)oxy)phenyl)-5-(4-fluorophenyl)-6-oxo-2,3,5,6-tetrahydrofuro[3,2-c]pyridine-7-carboxamide;
116) N-(3-fluoro-4-((6-methoxy-7-(3-(((tetrahydro-2H-pyran-4-yl)methyl)amino)propoxy)quinolin-4-yl)oxy)phenyl)-5-(4-fluorophenyl)-6-oxo-2,3,5,6-tetrahydrofuro[3,2-c]pyridine-7-carboxamide;
117) N-(3-fluoro-4-((7-(2-(3-hydroxyazetidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
118) N-(3-fluoro-4-((7-(2-(3-hydroxy-3-methylazetidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
119) N-(3-fluoro-4-((7-(2-(3-(hydroxymethyl)azetidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
120) N-(3-fluoro-4-((6-methoxy-7-(2-(3-methoxyazetidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
121) N-(3-fluoro-4-((6-methoxy-7-(2-(3-methoxy-3-methylazetidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
122) N-(3-fluoro-4-((6-methoxy-7-(2-(3-methoxymethyl)azetidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
123) N-(3-fluoro-4-((7-(2-(3-fluoroazetidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
124) N-(4-((7-(2-(3-(3,3-difluoroazetidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
125) N-(4-((7-(2-(3-(3-ethynylazetidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
126) N-(3-fluoro-4-((6-methoxy-7-(2-(3-methylazetidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
127) N-(4-((7-(2-(3-(3,3-dimethylazetidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
128) N-(4-((7-(2-(3-(dimethylamino)azetidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
129) N-(3-fluoro-4-((7-(2-(3-hydroxypyrrolidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
130) N-(3-fluoro-4-((7-(2-(3-hydroxy-3-methylpyrrolidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
131) N-(3-fluoro-4-((6-methoxy-7-(2-(3-methoxypyrrolidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
132) N-(3-fluoro-4-((6-methoxy-7-(2-(pyrrolidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
133) N-(3-fluoro-4-((7-(2-(3-fluoropyrrolidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
134) N-(4-((7-(2-(3,3-difluoropyrrolidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
135) N-(3-fluoro-4-((7-(2-(4-hydroxypiperidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
136) N-(3-fluoro-4-((7-(2-(3-hydroxypiperidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
137) N-(3-fluoro-4-((7-(2-(4-hydroxy-4-methylpiperidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
138) N-(3-fluoro-4-((6-methoxy-7-(2-(4-methoxypiperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
139) N-(3-fluoro-4-((6-methoxy-7-(2-(3-methoxypiperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
140) N-(3-fluoro-4-((6-methoxy-7-(2-(4-oxopiperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
141) N-(3-fluoro-4-((6-methoxy-7-(2-(3-oxopiperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
142) N-(4-((7-(2-(1,1-dioxydothiomorpholino)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
143) N-(3-fluoro-4-((6-methoxy-7-(2-(piperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
144) N-(3-fluoro-4-((7-(2-(4-fluoropiperidin-1-yl)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
145) N-(4-((7-(2-(4,4-difluoropiperidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
146) N-(3-fluoro-4-((6-methoxy-7-(2-(4-methylpiperidin-1-yl)ethoxy)quinolin-4-yl)oxy)phenyl)-5-(4-fluorophenyl)-6-oxo-2,3,5,6-tetrahydrofuro[3,2-c]pyridine-7-carboxamide;
147) N-(4-((7-(2-(4,4-dimethylpiperidin-1-yl)ethoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
148) N-(3-fluoro-4-((7-(2-((3-hydroxycyclobutyl)amino)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
149) N-(3-fluoro-4-((6-methoxy-7-(2-((3-methoxycyclobutyl)amino)ethoxy)quinolin-4-yl)oxy)phenyl)-5-(4-fluorophenyl)-6-oxo-2,3,5,6-tetrahydrofuro[3,2-c]pyridine-7-carboxamide;
150) N-(3-fluoro-4-((6-methoxy-7-(2-(oxetan-3-ylamino)ethoxy)quinolin-4-yl)oxy)phenyl)-5-(4-fluorophenyl)-6-oxo-2,3,5,6-tetrahydrofuro[3,2-c]pyridine-7-carboxamide;
151) N-(3-fluoro-4-((6-methoxy-7-(2-((oxetan-3-ylmethyl)amino)ethoxy)quinolin-4-yl)oxy)phenyl)-5-(4-fluorophenyl)-6-oxo-2,3,5,6-tetrahydrofuro[3,2-c]pyridine-7-carboxamide;
152) N-(3-fluoro-4-((7-(2-((4-hydroxycyclohexyl)amino)ethoxy)-6-methoxyquinolin-4-yl)oxy)phenyl)-5-(4-fluorophenyl)-6-oxo-2,3,5,6-[3,2-c]pyridine-7-carboxamide;
153) N-(3-fluoro-4-((7-(2-((3-hydroxycyclohexyl)amino)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
154) N-(3-fluoro-4-((7-(2-(((3-hydroxycyclohexyl)methyl)amino)ethoxy)-6-methoxyquinolin-4-yl)oxy)phenyl)-5-(4-fluorophenyl)-6-oxo-2,3,5,6-tetrahydrofuro[3,2-c]pyridine-7-carboxamide;
155) N-(3-fluoro-4-((6-methoxy-7-(2-((tetrahydro-2H-pyran-4-yl)amino)ethoxy)quinolin-4-yl)oxy)phenyl)-5-(4-fluorophenyl)-6-oxo-2,3,5,6-tetrahydrofuro[3,2-c]pyridine-7-carboxamide;
156) N-(3-fluoro-4-((6-methoxy-7-(2-((4-methoxycyclohexyl)amino)ethoxy)quinolin-4-yl)oxy)phenyl)-5-(4-fluorophenyl)-6-oxo-2,3,5,6-tetrahydrofuro[3,2-c]pyridine-7-carboxamide;
157) N-(3-fluoro-4-((7(2-morpholinylethoxy)quinolin-4-yl)oxy)phenyl)-5-(4-fluorophenyl)-6-oxo-2,3,5,6-tetrahydrofuro[3,2-c]pyridine-7-carboxamide;
158) N-(3-fluoro-4-((7-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
159) N-(3-fluoro-4-((7-(3-morpholinopropoxy)quinolin-4-yl)oxy)phenyl)-2-(4-fluorophenyl)-3-oxo-3,5,6,7-tetrahydro-2H-cyclopenta[c]pyridin-4-carboxamide;
160) N-(3-fluoro-4-((7-(3-(4-methylpiperazin-1-yl)propoxy)quinolin-4-yl)oxy)phenyl)-5-(4-fluorophenyl)-6-oxo-2,3,5,6-tetrahydrofuro[3,2-c]pyridine-7-carboxamide;
161) 7-(2-(3-fluoro-4-((7-(3-(piperidin-1-yl)propoxy)quinolin-4-yl)oxy)phenyl)acetyl)-5-(4-fluorophenyl)-3,5-dihydrofuro[3,2-c]pyridin-6(2H)-one;
162) 7-(2-(3-fluoro-4-((7-(3-(4-methylpiperidin-1-yl)propoxy)quinolin-4-yl)oxy)phenyl)acetyl)-5-(4-fluorophenyl)-3,5-dihydrofuro[3,2-c]pyridin-6(2H)-one;
163) N-(3-fluoro-4-((6-(methylcarbamoyl)-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
164) N-(3-fluoro-4-((6-(methylcarbamoyl)-7-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
165) N-(3-fluoro-4-((6-methoxy-7-(methylcarbamoyl)quinolin-4-yl)oxy)phenyl)-5-(4-fluorophenyl)-6-oxo-2,3,5,6-tetrahydrofuro[3,2-c]pyridine-7-carboxamide;
166) N-(3-fluoro-4-((7-(methylcarbamoyl)-6-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
167) N-(3-fluoro-4-((6-fluoro-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
168) N-(3-fluoro-4-((6-fluoro-7-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
169) N-(3-fluoro-4-((7-(2-morpholinoethoxy)-6-(trifluoromethyl)quinolin-4-yl)oxy)phenyl)-5-(4-fluorophenyl)-6-oxo-2,3,5,6-tetrahydrofuro[3,2-c]pyridine-7-carboxamide;
170) N-(3-fluoro-4-((7-(3-morpholinopropoxy)-6-(trifluoromethyl)quinolin-4-yl)oxy)phenyl)-5-(4-fluorophenyl)-6-oxo-2,3,5,6-tetrahydrofuro[3,2-c]pyridine-7-carboxamide;
171) N-4-((6-chloro-7-(2-morpholinoethoxy)quinolin-4-yl)oxy)-3-fluorophenyl)-5-(4-fluorophenyl)-6-oxo-2,3,5,6-tetrahydrofuro[3,2-c]pyridine-7-carboxamide;
172) N-4-((6-chloro-7-(3-morpholinopropoxy)quinolin-4-yl)oxy)-3-fluorophenyl)-5-(4-fluorophenyl)-6-oxo-2,3,5,6-tetrahydrofuro[3,2-c]pyridine-7-carboxamide;
173) N-(4-((6-cyano-7-(2-morpholinoethoxy)quinolin-4-yl)oxy)-3-fluorophenyl)-5-(4-fluorophenyl)-6-oxo-2,3,5,6-tetrahydrofuro[3,2-c]pyridine-7-carboxamide;
174) N-(4-((6-cyano-7-(3-morpholinopropoxy)quinolin-4-yl)oxy)-3-fluorophenyl)-5-(4-fluorophenyl)-6-oxo-2,3,5,6-tetrahydrofuro[3,2-c]pyridine-7-carboxamide;
175) N-(3-fluoro-4-((7-methoxy-6-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
176) N-(3-fluoro-4-((7-methoxy-6-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
177) N-(3-fluoro-4-((6-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide;
178) N-(3-fluoro-4-((6-(3-morpholinopropoxy)quinolin-4-yl)oxy)phenyl)-5-(4-fluorophenyl)-6-oxo-2,3,5,6-tetrahydrofuro[3,2-c]pyridine-7-carboxamide;
179) N-(4-((6-amino-7-methoxyquinolin-4-yl)oxy)-3-fluorophenyl)-5-(4-fluorophenyl)-6-oxo-2,3,5,6-tetrahydrofuro[3,2-c]pyridine-7-carboxamide;
180) N-(3-fluoro-4-((7-methoxy-6-(3-morpholinopropanamido)quinolin-4-yl)oxy)phenyl)-5-(4-fluorophenyl)-6-oxo-2,3,5,6-tetrahydrofuro[3,2-c]pyridine-7-carboxamide;
181) N-(4-((7-amino-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
182) N-(3-fluoro-4-((6-methoxy-7-(2-morpholinoacetamido)quinolin-4-yl)oxy)phenyl)-5-(4-fluorophenyl)-6-oxo-2,3,5,6-tetrahydrofuro[3,2-c]pyridine-7-carboxamide;
183) N-(3-fluoro-4-((6-methoxy-7-(3-morpholinopropanamido)quinolin-4-yl)oxy)phenyl)-5-(4-fluorophenyl)-6-oxo-2,3,5,6-tetrahydrofuro[3,2-c]pyridine-7-carboxamide;
184) N-(4-((7-acetamido-6-methoxyquinolin-4-yl)oxy)-3-fluorophenyl)-5-(4-fluorophenyl)-6-oxo-2,3,5,6-tetrahydrofuro[3,2-c]pyridine-7-carboxamide;
185) N-(3-fluoro-4-((7-methoxy-6-((2-morpholinoethyl)amino)quinolin-4-yl)oxy)phenyl)-5-(4-fluorophenyl)-6-oxo-2,3,5,6-tetrahydrofuro[3,2-c]pyridine-7-carboxamide;
186) N-(3-fluoro-4-((7-methoxy-6-((3-morpholinopropyl)amino)quinolin-4-yl)oxy)phenyl)-5-(4-fluorophenyl)-6-oxo-2,3,5,6-tetrahydrofuro[3,2-c]pyridine-7-carboxamide;
187) N-(3-fluoro-4-((6-methoxy-7-((2-morpholinoethyl)amino)quinolin-4-yl)oxy)phenyl)-5-(4-fluorophenyl)-6-oxo-2,3,5,6-tetrahydrofuro[3,2-c]pyridine-7-carboxamide; and
188) N-(3-fluoro-4-((6-methoxy-7-((3-morpholinopropyl)amino)quinolin-4-yl)oxy)phenyl)-5-(4-fluorophenyl)-6-oxo-2,3,5,6-tetrahydrofuro[3,2-c]pyridine-7-carboxamide.

Preferably, the compound represented by Formula 2 above may be:
N-(3-fluoro-4-((6-methoxy-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide; or
N-(4-((7-(3-(3-cyanoazetidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide.

Hereinafter, a method for preparing the compound of Formula 2 above will be described. The compound of Formula 2 above can be prepared using a method as shown by the following reaction schemes. However, the present invention is not limited to a case where the compound is prepared by this method. In particular, those skilled in the art will fully appreciate that the compounds of Formula 2 of the present invention can be prepared by a variety of methods using the techniques well known in the art.

The following reaction schemes show, in respective preparation steps, a method for preparing the compound of Formula 2 above of the present invention, and various compounds of Formula 2 above may be prepared by changing reagents and solvents used in the following preparation steps or by changing the order of reactions.

According to an embodiment, the compound of Formula 2 may be prepared according to the procedures in Reaction Schemes 1 and 2.

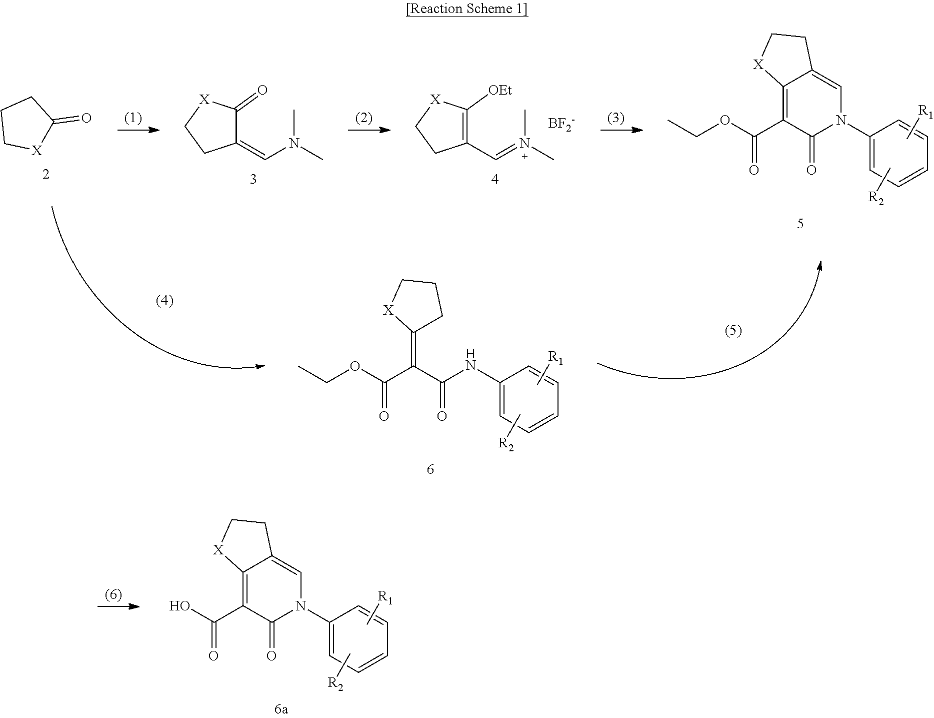

In Reaction Scheme 1 above, R₁, R₂, and X are as defined in Formula 2 above.

According to Reaction Scheme 1, a carboxylic acid compound (6a) is prepared using, as a starting material, a lactone-based compound (2) that can be easily obtained commercially or is prepared by a known method.

Regarding Reaction Scheme 1, detailed description of each step is as follows.

In step 1, a compound (2), which can be easily obtained commercially, is subjected to formylation reaction using dimethyldimethoxyacetal, to prepare a compound of Formula (3).

The reaction may generally be carried out at a high temperature; however, this is disadvantageous in that a long reaction time is required. Thus, the reaction is carried out using a microwave reactor.

In step 2, a compound (4) is prepared using the formylated lactone compound (3) in step 1 and triethyloxonium tetrafluoroborate which can be easily obtained commercially. This reaction is carried out under anhydrous conditions and is preferably carried out using a solvent, which does not adversely affect the reaction, such as N,N-dichloromethane or chloroform. For the reaction temperature, the reaction is generally carried out at room temperature.

In step 3, the compound (4) prepared in step 2 is reacted with an ethyl-3-amino-3-oxopropionate compound, which has been prepared by a known method, in the presence of sodium ethoxide to prepare a cyclized compound (5). This reaction is preferably carried out using an ethanol solvent that does not adversely affect the reaction. The reaction temperature is not particularly limited. The reaction may generally be carried out at from a cold temperature to a warm temperature, and is preferably carried out at room temperature.

Alternatively, to prepare the cyclized compound (5), as in step 4, the lactone-based compound (2), which can be easily obtained commercially and is used as a starting material, may be reacted with an ethyl-3-amino-3-oxopropionate compound, which has been prepared by a known method, in the presence of titanium tetrachloride and pyridine to prepare a compound (6). This reaction is preferably carried out using dichloromethane that does not adversely affect the reaction. The reaction temperature is not particularly limited; and the reaction may generally be carried out at a cold to room temperature, and preferably starting from a cold temperature up to room temperature.

Thereafter, in step 5, the compound (6) prepared in step 4 is subjected to formylation and cyclization using dimethyldimethoxyacetal to prepare the compound (5). The reaction may be generally carried out at a warm and high temperature, and is preferably carried out at a warm temperature.

In step 6, the cyclized compound (5) prepared in steps 3 and 5 is subjected to hydrolysis, to prepare the carboxylic acid compound (6a). In general, the hydrolysis is carried out using a basic aqueous solution such as an aqueous sodium hydroxide solution or an aqueous lithium hydroxide solution. This reaction is carried out using a solvent, which does not adversely affect the reaction, such as ethanol, methanol, or tetrahydrofuran, in the presence of an aqueous lithium hydroxide solution that can be used in the hydrolysis. The reaction temperature is not particularly limited. The reaction may generally be carried out at room temperature or a warm temperature, preferably at a warm temperature to prepare the carboxylic acid compound (6a).

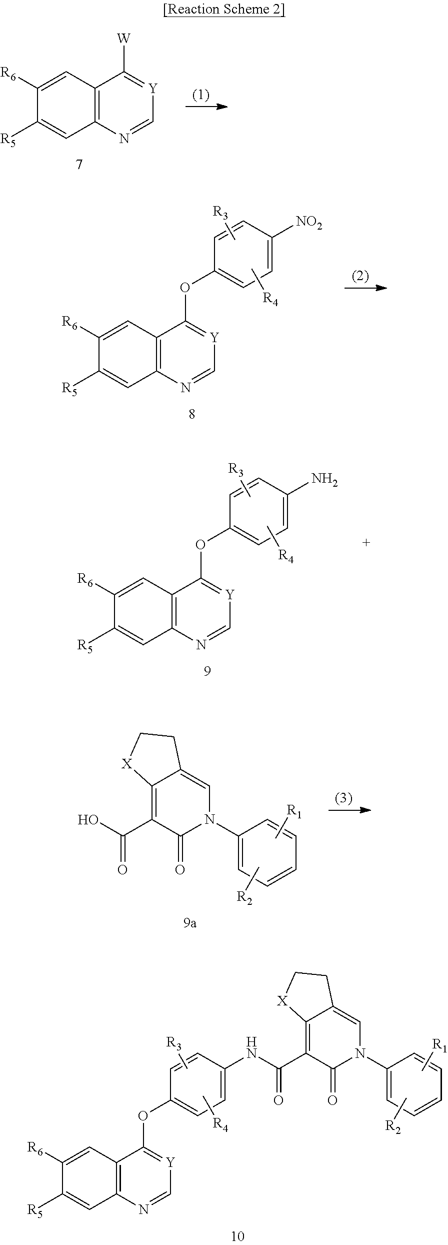

In Reaction Scheme 2, R₁ to R₆, X, and Y are as defined in Formula 2 above, and W is a leaving group.

Reaction Scheme 2 specifically shows each step for preparing a desired compound (10) of the present invention.

In step 1, a monocyclic or bicyclic compound (7), which can be easily obtained commercially or prepared by a known method, is reacted with a nitrophenol compound, which can be easily obtained commercially, in the presence of a base such as potassium carbonate, to obtain a phenoxy compound (8). This reaction is a general ether-forming reaction of a phenolic compound, and is carried out in the presence of a base which can be used for the ether-forming reaction. Examples of the base that can be used for this purpose include sodium hydrate (NaH), potassium carbonate, sodium carbonate, cesium carbonate, sodium or potassium alkoxide, and the like. In addition, the reaction is preferably carried out in the presence of a solvent that does not adversely affect the reaction. For example, the reaction is carried out using a solvent such as dichloromethane, chloroform, tetrahydrofuran, diethyl ether, toluene, N,N-dimethylformamide, acetonitrile, or diphenyl ether. The reaction temperature is not particularly limited. The reaction may generally be carried out at room temperature or a warm temperature, preferably at a warm temperature.

In step 2, the nitrophenol compound (8) prepared in step 1 is subjected to reduction in the presence of iron and ammonium chloride, to prepare an amine compound (9). This reaction is generally reduction of a nitro compound into an amine, and may be carried out using various reducing agents such as hydrogen, iron, tin(II) chloride, and zinc. In addition, for this reaction, a solvent, which does not adversely affect the reaction, is used, such as dichloromethane, ethyl acetate, methanol, ethanol, tetrahydrofuran, or N,N-dimethylformamide. As the case may be, the reaction is carried out using water as a co-solvent. The reaction temperature is not particularly limited. The reaction may generally be carried out at room temperature or a warm temperature, preferably at a warm temperature.

In step 3, a common amidation reaction is carried out, in which the amine compound (9) prepared in step 2 is reacted with the carboxylic acid compound (6a) prepared in Reaction Scheme 1 using a coupling reagent to prepare the desired compound (10). In general, the reaction is carried out using a coupling reagent, which can be easily obtained commercially, such as 1-(3-dimethylaminopropyl)-3-ethylcarbodiimide (EDC), 1,3-dicyclohexyl carbodiimide (DCC), 1,1-carbonyl diimidazole (CDI), or 1-[bis((dimethylamino)methylene]-1H-1,2,3-triazolo[4,5-b]pyridinium 3-oxide hexafluorophosphate (HATU). This reaction may be carried out without using a base; however, the reaction is carried out using a solvent, which does not adversely affect the reaction, such as acetonitrile, dimethylformamide, or dichloromethane, in the presence of a common base, which can be used for an amidation reaction, such as 4-dimethylaminopyridine, pyridine, triethylamine, diethylisopropylamine, N-methylmorpholine, or dimethylphenylamine. The reaction temperature is not particularly limited. The reaction may generally be carried out at room temperature or a warm temperature, preferably at a warm temperature to prepare the desired compound (10).

The desired compounds produced in the schemes can be separated and purified using conventional methods, for example, column chromatography, recrystallization, and the like.

As used herein, the term “halogen” refers to F, Cl, Br, or I unless stated otherwise.

The term “alkyl,” unless otherwise specified, refers to a linear or branched saturated hydrocarbon radical. For example, “C₁₋₁₀ alkyl” refers to an alkyl having a skeleton formed of 1 to 10 carbon atoms. Specifically, C₁₋₁₀alkyl may include methyl, ethyl, n-propyl, i-propyl, n-butyl, i-butyl, t-butyl, n-pentyl, i-pentyl, t-pentyl, sec-pentyl, neopentyl, hexyl, heptyl, octyl, nonyl, decyl, and the like.

The term “haloalkyl” refers to an alkyl substituted with one or more halogen atoms. Specifically, haloalkyl may be an alkyl substituted with two or more halogen atoms of the same kind or substituted with two or more kinds of halogen atoms.

The term “alkoxy”, unless otherwise specified, refers to a group having the formula —O— alkyl, in which the alkyl is an alkyl group as defined above and is attached to a parent compound through an oxygen atom. The alkyl moiety in the alkoxy group may have 1 to 20 carbon atoms (that is, C₁-C₂₀ alkoxy), 1 to 12 carbon atoms (that is, C₁-C₁₂ alkoxy), or 1 to 6 carbon atoms (that is, C₁-C₆ alkoxy). Examples of suitable alkoxy groups include methoxy (—O—CH₃ or —OMe), ethoxy (—OCH₂CH₃ or -OEt), t-butoxy (—O—C(CH₃)₃ or —O-tBu), and the like.

The term “aryl” refers to an aromatic hydrocarbon radical derived by the removal of one hydrogen atom from a single carbon atom of a parent aromatic ring system. For example, the aryl group may have 6 to 20 carbon atoms, 6 to 14 carbon atoms, or 6 to 10 carbon atoms.

The term “cycloalkyl” refers to a saturated monocycle or polycycle that contains only carbon atoms in the ring. The cycloalkyl may have 3 to 7 carbon atoms as a monocycle, 7 to 12 carbon atoms as a bicycle, and up to about 20 carbon atoms as a polycycle.

The term “heteroaryl” refers to aromatic heterocyclyl having one or more heteroatoms in the ring. Non-limiting examples of the heteroaryl include pyridinyl, pyrrolyl, oxazolyl, indolyl, isoindolyl, furinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, and the like (each of which may have one or more substituents on the ring).

The term “heterocycle” refers to an aromatic or non-aromatic ring having one or more heteroatoms, which may be saturated or unsaturated and may be monocyclic or polycyclic. For example, “4- to 10-membered heterocycle” means a heterocycle comprising a total of 4 to 10 atoms constituting the skeleton, including heteroatom(s) and carbon atoms. Specifically, examples of the 4- to 10-membered heterocycle may include azetidine, diazetidine, pyrrolidine, pyrrole, imidazolidine, imidazole, pyrazolidine, pyrazole, oxazolidine, oxazole, isoxazolidine, isoxazole, thiazolidine, thiazole, isothiazolidine, isothiazole, piperidine, pyridine, piperazine, diazine, morpholine, thiomorpholine, azepane, diazepane, and the like.

The term “heterocycloalkyl” refers to a non-aromatic heterocyclyl having one or more heteroatoms in the ring. Heterocycloalkyl may have one or more carbon-carbon double bonds or carbon-heteroatom double bonds in the ring to the extent that the ring does not become aromatic due to presence of the double bond. Non-limiting examples of heterocycloalkyl include azetidinyl, aziridinyl, pyrrolidinyl, piperidinyl, piperazinyl, homopiperazinyl, morpholino, thiomorpholino, tetrahydrofuranyl, tetrahydrothiofuranyl, tetrahydropyranyl, pyranyl (each of which may have one or more substituents on the ring), and the like.

The term “heteroatom” refers to an atom other than carbon (C), specifically nitrogen (N), oxygen (O), or sulfur (S) atom. The above-mentioned heteroaryl and heterocycloalkyl contain one or more heteroatoms and may, for example, contain 1, 1 to 2, 1 to 3, or 1 to 4 heteroatoms.

The term “substitution” refers to replacing a hydrogen atom in a molecular structure with a substituent, such that the valence on the designated atom is not exceeded, and such that a chemically stable compound results from the substitution. For example, “group A is substituted with substituent B” or “group A has substituent B” means that a hydrogen atom bonded to an atom, such as carbon, which constitutes a skeleton of group A, is replaced with substituent B so that the group A and the substituent B form a covalent bond. Thus, it is substantially difficult or impossible for a group having no removable hydrogen atom to have a substituent. From this viewpoint, in a case where a range of combinations of various groups, which include groups that hardly have a substituent, with substituents are exemplified in the present specification, it should be interpreted that combinations of the groups, for which it is obvious that no substitution is possible, with the substituents are excluded from the range.

The pharmaceutical composition of the present invention includes, as an active ingredient, a pharmaceutically acceptable salt of the compound represented by Formula 1 or Formula 2 above.

The pharmaceutically acceptable salt should have low toxicity to humans and should not have any negative effect on biological activity and physicochemical properties of a parent compound.

For example, the pharmaceutically acceptable salt may be an acid addition salt formed with a pharmaceutically acceptable free acid.

As the free acid, an inorganic acid or an organic acid may be used, in which the inorganic acid may be hydrochloric acid, sulfuric acid, nitric acid, phosphoric acid, perchloric acid, bromic acid, or the like, and the organic acid may be acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid, glutamic acid, or the like.

The acid addition salt may be prepared by a conventional method, for example, by dissolving the compound of Formula 1 or Formula 2 in an excess of aqueous acid solution, and precipitating the salt using a water-miscible organic solvent such as methanol, ethanol, acetone, or acetonitrile.

In addition, the pharmaceutically acceptable salt may be an alkali metal salt (such as sodium salt) or an alkaline earth metal salt (such as potassium salt).

The alkali metal salt or alkaline earth metal salt may be obtained, for example, by dissolving the compound of Formula 1 or Formula 2 in an excess of alkali metal hydroxide or alkaline earth metal hydroxide solution, filtering the undissolved compound salt, and then evaporating and drying the filtrate.

In addition, the compound of the present invention may have a chiral carbon center, and thus may exist in the form of R or S isomers, racemic compounds, individual enantiomers or mixtures thereof, individual diastereomers or mixtures thereof. All these stereoisomers and mixtures thereof may fall within the scope of the present invention.

In addition, the compound of the present invention may include hydrates and solvates of the compound of Formula 1 or Formula 2. The hydrates and solvates may be prepared using known methods, and are preferably non-toxic and water-soluble. In particular, the hydrates and solvates may preferably be formed by being combined with 1 to 5 molecules of water and alcoholic solvent (in particular, ethanol, or the like), respectively.

The pharmaceutical composition of the present invention may contain, as an active ingredient, the compound represented by Formula 1, or a pharmaceutically acceptable salt thereof, in an amount of about 0.1% to about 90% by weight, specifically about 0.5% by weight to about 75% by weight, and more specifically about 1% by weight to about 50% by weight, based on the total weight of the composition.

The pharmaceutical composition of the present invention may contain conventional, non-toxic pharmaceutically acceptable additives which are combined into preparations according to conventional methods. For example, the pharmaceutical composition may further contain a pharmaceutically acceptable carrier, diluent, or excipient.

Examples of additives used in the composition of the present invention may include sweeteners, binders, solvents, solubilizers, wetting agents, emulsifiers, isotonic agents, absorbents, disintegrants, antioxidants, preservatives, lubricants, glidants, fillers, flavors, and the like. For example, the additives may include lactose, dextrose, sucrose, mannitol, sorbitol, cellulose, glycine, silica, talc, stearic acid, stearin, magnesium stearate, magnesium aluminosilicate, starch, gelatin, tragacanth gum, alginic acid, sodium alginate, methyl cellulose, sodium carboxymethyl cellulose, agar, water, ethanol, polyethylene glycol, polyvinylpyrrolidone, sodium chloride, calcium chloride, orange essence, strawberry essence, vanilla flavor, and the like.

The composition of the present invention may be formulated in various preparation forms for oral administration (for example, tablets, pills, powders, capsules, syrups, or emulsions) or parenteral administration (for example, intramuscular, intravenous, or subcutaneous injection).

Preferably, the composition of the present invention may be formulated into preparations for oral administration. Examples of the additives for this purpose may include cellulose, calcium silicate, com starch, lactose, sucrose, dextrose, calcium phosphate, stearic acid, magnesium stearate, calcium stearate, gelatin, talc, surfactants, suspending agents, emulsifying agents, diluents, and the like. Specifically, examples of the glidant include colloidal silicon dioxide, magnesium silicate, and the like; examples of the diluent include microcrystalline cellulose, Fast Flo® lactose, lactose anhydrous, lactose monohydrate, silicified MCC HD 90, and the like; examples of the disintegrant include croscarmellose sodium, crospovidone, and the like; and examples of the lubricant include magnesium stearate, sodium lauryl sulfate, stearic acid, and the like.

In addition, as liquid preparations for oral administration, suspensions, emulsions, syrups, and the like may be exemplified, and the liquid preparations may contain various excipients such as wetting agents, sweeteners, fragrances, preservatives, and the like, in addition to water and liquid paraffin which are commonly used simple diluents.

In addition, examples of preparations for parenteral administration may include sterilized aqueous solutions, non-aqueous solutions, suspensions, emulsions, freeze-dried preparations, and suppositories. For the non-aqueous solutions and the suspensions, propylene glycol, polyethylene glycol, vegetable oil such as olive oil, injectable ester such as ethyl oleate, or the like may be used. As the suppository base, Witepsol™, macrogol, Tween™ 61, cacao butter, laurin fat, glycerogelatin, or the like may be used. On the other hand, injections may contain conventional additives such as solubilizers, isotonic agents, suspending agents, emulsifiers, stabilizers, and preservatives.

The term “prevention” refers to all actions that inhibit cholangiocarcinoma or delay the onset thereof by the administration of the pharmaceutical composition. The term “prevention” refers to all actions in which symptoms of cholangiocarcinoma are ameliorated or beneficially changed by the administration of the pharmaceutical composition.

The compound or composition of the present invention may be administered to a patient in a therapeutically effective amount or in a pharmaceutically effective amount.

As used herein, the term “therapeutically effective amount” or “pharmaceutically effective amount” refers to an amount of a compound or composition effective to prevent or treat the disease in question, which is sufficient to treat the disease at a reasonable benefit/risk ratio applicable to medical treatment and does not cause adverse effects. The level of the effective amount may be determined depending on factors including the patient's health condition, type and severity of disease, activity of drug, the patient's sensitivity to drug, mode of administration, time of administration, route of administration and excretion rate, duration of treatment, formulation or simultaneously used drugs, and other factors well known in the medical field.

The term “administration” means introducing a predetermined drug to a patient by any appropriate method, and the drug may be administered through any general route as long as the drug may reach a target tissue. Such a route of administration may include, but is not limited to, intraperitoneal administration, intravenous administration, intramuscular administration, subcutaneous administration, intradermal administration, oral administration, topical administration, intranasal administration, intrapulmonary administration, intrarectal administration, and the like.

The compound or composition of the present invention may be administered as an individual therapeutic agent or in combination with other therapeutic agents, may be administered sequentially or simultaneously with a conventional therapeutic agent, and may be administered in single or multiple doses. It is important that taking into account all of the above factors, the amount which is a minimum amount that allows a maximum effect to be obtained without adverse effects is administered, and such an amount may be easily determined by those skilled in the art.

Specifically, the effective amount of the compound in the composition of the present invention may vary depending on the patient's age, sex, and body weight; and in general, about 0.1 mg to about 1,000 mg or about 5 mg to about 200 mg per kg body weight may be administered daily or every other day, or once to three times a day. However, the effective amount may be increased or decreased depending on route of administration, severity of disease, the patient's sex, body weight, age, and the like, and thus the scope of the present invention is not limited thereto.

Preferably, the compound or composition of the present invention may be administered for tumor therapy in combination with chemotherapy, radiation therapy, immunotherapy, hormone therapy, bone marrow transplantation, stem cell replacement therapy, other biological therapies, surgical intervention, or combinations thereof. For example, the compound or composition of the present invention may be used as adjunctive therapy in conjunction with other long-term treatment strategies, or may be used to maintain the patient's condition after tumor regression or chemoprophylactic therapy in severe patients.

The pharmaceutical composition of the present invention may further contain at least one active ingredient, and examples of the further contained active ingredient may include, but are not limited to, anti-proliferative compounds such as aromatase inhibitors, anti-estrogens, topoisomerase I inhibitors, topoisomerase II inhibitors, microtubule active compounds, alkylating compounds, histone deacetylase inhibitors, compounds that induce cell differentiation processes, cyclooxygenase inhibitors, MMP inhibitors, mTOR inhibitors, anti-neoplastics, anti-metabolites, platin compounds, compounds that target/decrease activity of protein or lipid kinase, anti-angiogenic compounds, compounds that target, decrease, or inhibit activity of protein or lipid phosphatase, gonadorelin agonists, anti-androgens, methionine aminopeptidase inhibitors, bisphosphonates, biological response modifiers, anti-proliferative antibodies, heparanase inhibitors, Ras oncogenic isotype inhibitors, telomerase inhibitors, proteasome inhibitors, compounds used for treatment of hematologic malignancies, compounds which target, decrease, or inhibit activity of Flt-3, Hsp90 inhibitors, kinesin spindle protein inhibitors, MEK inhibitors, leucovorin, EDG binding agents, anti-leukemia compounds, ribonucleotide reductase inhibitors, S-adenosylmethionine decarboxylase inhibitors, hemostatic steroids, corticosteroids, other chemotherapeutic compounds, and photosensitizing compounds.

The further contained active ingredient may be a known anticancer drug. Non-limiting examples of the anticancer drug include DNA alkylating agents such as mechlorethamine, chlorambucil, phenylalanine, mustard, cyclophosphamide, ifosfamide, carmustine (BCNU), lomustine (CCNU), streptozotocin, busulfan, thiotepa, cisplatin, and carboplatin; anticancer antibiotics such as dactinomycin (actinomycin D), doxorubicin (adriamycin), daunorubicin, idarubicin, mitoxantrone, plicamycin, mitomycin C, and bleomycin; and plant alkaloids such as vincristine, vinblastine, paclitaxel, docetaxel, etoposide, teniposide, topotecan, and irinotecan; and the like.

In this case, the drug may be the compound represented by Formula 1 or Formula 2, or a pharmaceutically acceptable salt thereof.

Here, the meaning of exhibiting resistance to the EGFR-targeted therapeutic agent is as described above. In addition, the EGFR-targeted therapeutic agent may be at least one selected from the group consisting of cetuximab, gefitinib, erlotinib, apatinib, icotinib, brigatinib, lapatinib, canertinib, AEE788, XL647, zactima, and panitumumab.

In another aspect of the present invention, there is provided a use of the pharmaceutical composition for preventing and treating cholangiocarcinoma. In this case, the pharmaceutical composition is the same as described above.

In yet another aspect of the present invention, there is provided a use of the pharmaceutical composition for preparing a medicament for preventing or treating cholangiocarcinoma. In this case, the pharmaceutical composition is the same as described above.

In still another aspect of the present invention, there is provided a method for preventing or treating cholangiocarcinoma, the method including the steps of: detecting RON mutation in a biological sample derived from an individual suffering from cholangiocarcinoma, wherein the RON mutation is RONΔ155 in which exons 5, 6, and 11 are deleted, RONΔ160 in which exons 5 and 6 are deleted, or RONΔ165 in which exon 11 is deleted; and administering the pharmaceutical composition according to an aspect of the present invention to the individual in which the RON mutation has been detected.

The RON, RON mutation, pharmaceutical composition, administration, prevention, and treatment are the same as described above.

The biological sample refers to a sample obtained from the individual. The biological sample may be tissue, blood, plasma, serum, bone marrow fluid, lymph fluid, saliva, tear fluid, mucosal fluid, amniotic fluid, or a combination thereof.

The individual may be a mammal, such as a human, cow, horse, pig, dog, sheep, goat or cat. The individual may be a patient suffering from a disease associated with the mutation in RON, for example, cholagiocarcinoma, or an individual with a high likelihood of suffering from cholagiocarcinoma.

The method may further include administering an anticancer agent to the individual. The anticancer agent may be administered simultaneously, separately, or sequentially with the pharmaceutical composition.

The administration method may be oral or parenteral administration. The administration method may be, for example, oral, transdermal, subcutaneous, rectal, intravenous, intraarterial, intraperitoneal, intramuscular, intrasternal, topical, intranasal, intratracheal, or intradermal route. The composition may be administered systemically or locally, alone or in combination with other pharmaceutically active compounds.

The preferred dosage of the pharmaceutical composition varies with the condition and body weight of a patient, the severity of the disease, the form of drug, the route and duration of administration, but may be appropriately selected by one of ordinary skill in the art.

The dosage may be, for example, in the range of about 0.001 mg/kg to about 100 mg/kg, about 0.01 mg/kg to about 10 mg/kg, or about 0.1 mg/kg to about 1 mg/kg, based on an adult. The administration may be performed once a day, multiple times a day, or once a week, once every two weeks, once every three weeks, or once every four weeks to once a year.

In still yet another aspect of the present invention, there is provided a method for providing information on an anticancer therapeutic agent, the method including the steps of: detecting RON mutation in a biological sample derived from an individual suffering from cholangiocarcinoma, wherein the RON mutation is RONΔ155 in which exons 5, 6, and 11 are deleted, RONΔ160 in which exons 5 and 6 are deleted, or RONΔ165 in which exon 11 is deleted; and providing, to the individual in which the RON mutation has been detected, information that the pharmaceutical composition according to an aspect of the present invention is suitable for preventing or treating cholangiocarcinoma.

As used herein, the term “suitable for anticancer therapy” means that it can be used for anticancer therapy, and the phrase “providing information that it is suitable for anticancer therapy” means providing information through which it is possible to determine whether or not it can be selected as a drug that can be used for anticancer therapy.

The biological sample, RON, RON mutation, pharmaceutical composition, individual, prevention, and treatment are the same as described above.

### MODE FOR THE INVENTION

Hereinafter, the present invention will be described in more detail by way of the following examples. However, the following examples are only provided for easier understanding of the present invention, and the scope of the present invention is not limited thereto.

### Preparation Example 1: 5-(4-Fluorophenyl)-6-oxo-2,3,5,6-tetrahydrofuro[3,2-c]pyridine-7-carboxylic acid

### Step 1: Synthesis of 3-((dimethylamino)methylene)-dihydrofuran-2-(3H)-one

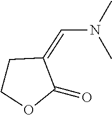

Gamma-butyrolactone (2.69 mL, 35.0 mmol) and dimethyldimethoxyacetal (11.62 mL, 87.5 mmol) were stirred, and then reacted in a microwave reactor at a temperature of 230° C. for 70 minutes or longer. The reaction mixture was concentrated to remove excess dimethyldimethoxyacetal. Solidification was performed with diethyl ether, and the precipitated solid was filtered while washing with diethyl ether. The filtered solid was concentrated under reduced pressure to obtain the title compound (2.9 g, yield: 59%, brownish solid).

¹H NMR (500 MHz, CDCl₃) δ 7.13 (s, 1H), 4.24 (t, J=7.5 Hz, 2H), 3.11 (t, J=7.5 Hz, 2H), 3.03 (s, 6H); ¹H NMR (500 MHz, DMSO-d₆) δ 7.00 (t, J=1.5 Hz, 1H), 4.11 (t, J=7.5 Hz, 2H), 3.06 (t, J=7.5 Hz, 2H), 3.00 (s, 3H)

### Step 2: Synthesis of 3-((dimethylamino)methylene)-2-(3H)-dihydrofuranilidene ethyl oxonium tetrafluoroborate

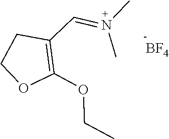

The compound (1.085 g, 7.68 mmol) obtained in step 1 was dissolved in 8 mL of chloroform. Then, triethyloxonium tetrafluoroborate (1.729 g, 7.68 mmol) was added thereto and stirred under nitrogen condition at room temperature for 1 day or longer. The reaction mixture was concentrated under reduced pressure. Nuclear magnetic resonance was used to identify that the starting material and the resulting material were produced in a ratio of about 15:85, and the next reaction was performed without purification.

¹H NMR (500 MHz, DMSO-d₆) δ 7.93 (s, 1H), 4.86-4.82 (m, 2H), 4.51 (q, J=7.0 Hz, 2H), 3.33 (s, 3H), 3.30 (t, J=8.5 Hz, 2H), 3.26 (s, 3H), 1.36 (t, J=7.0 Hz, 3H)

### Step 3: Synthesis of ethyl 5-(4-fluorophenyl)-6-oxo-2,3,5,6-tetrahydrofuro[3,2-c]pyridine-7-carboxylate

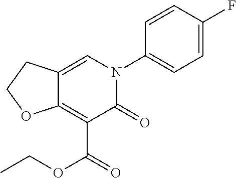 [SMILES: CCOC(=O)c1c2c(cn(-c3ccc(F)cc3)c1=O)CCO2]

The crude mixture (1.96 g) obtained in step 2 was dissolved in 10 mL of ethanol. Subsequently, sodium ethoxide (20 wt % solution in ethanol, 2.56 mL, 6.53 mmol) was added thereto in a water bath at 0° C., and then slowly stirred for 30 minutes to room temperature. To the reaction mixture was added ethyl 3-((4-fluorophenyl)amino)-3-oxopropionate (1.47 g, 6.53 mmol), and stirring was performed at room temperature for 20 hours or longer. The reaction mixture was concentrated under reduced pressure and extracted with water and dichloromethane. The separated organic layer was dried over anhydrous magnesium sulfate, filtered, and concentrated under reduced pressure. The resulting residue was purified by column chromatography to obtain the title compound (900 mg, yield: 39% (based on overall yield of steps 2 and 3)/46% (based on yield of step 3), yellow solid).

¹H NMR (500 MHz, DMSO-d₆) δ 7.70 (t, J=1.5 Hz, 1H), 7.42-7.40 (m, 2H), 7.34-7.31 (m, 2H), 4.74 (t, J=8.0 Hz, 2H), 4.17 (q, J=7.0 Hz, 2H), 3.05 (td, J=8.0 Hz, 2H), 1.21 (t, J=7.0 Hz, 3H)

### Step 4: Synthesis of 5-(4-fluorophenyl)-6-oxo-2,3,5,6-tetrahydrofuro[3,2-c]pyridine-7-carboxylic acid

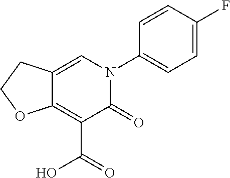 [SMILES: O=C(O)c1c2c(cn(-c3ccc(F)cc3)c1=O)CCO2]

The compound (0.9 g, 2.97 mmol) obtained in step 3 was dissolved in 10 mL of ethanol and 5 mL of distilled water, and then lithium hydroxide monohydrate (249 mg, 5.94 mmol) was added thereto. The mixture was heated to a temperature of 50° C. and stirred for 4 hours or longer. The reaction mixture was concentrated under reduced pressure and extracted with water and dichloromethane. 1 N hydrochloric acid solution was added to the separated water layer, and then the organic layer was extracted with water and dichloromethane. The separated organic layer was dried over anhydrous magnesium sulfate, filtered, and concentrated under reduced pressure. The concentrated residue was treated with a small amount of dichloromethane and diethyl ether to precipitate a solid, and filtration was performed. Then, the filtered solid was dried to obtain the title compound (680 mg, yield: 84%, off-white solid).

¹H NMR (500 MHz, DMSO-d₆) δ 14.5 (bs, OH), 7.97 (s, 1H), 7.54-7.51 (m, 2H), 7.41-7.37 (m, 2H), 4.90 (t, J=8.5 Hz, 2H), 3.11 (td, J=8.5, 1.0 Hz, 2H)

### Example 1: Synthesis of 4-Ethoxy-N-[3-fluoro-4-({2-[5-(morpholinomethyl)pyridin-2-yl]thieno[3,2-b]pyridin-7-yl}oxy)phenyl]-1-(4-fluorophenyl)-2-oxo-1,2-dihydropyridine-3-carboxamide hydrochloride

The title compound was synthesized by a method described in Example 31 of Korean Patent Laid-open Publication No. 2019-0106802 (Korean Patent No. 10-2221689). The title compound was named WM-S1-030.

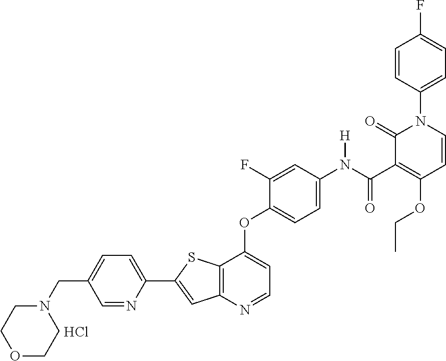 [SMILES: CCOc1ccn(-c2ccc(F)cc2)c(=O)c1C(=O)Nc1ccc(Oc2ccnc3cc(-c4ccc(CN5CCOCC5)cn4)sc23)c(F)c1 ; Cl]

¹H NMR (500 MHz, DMSO-d₆) δ 10.66 (s, 1H), 8.81 (s, 1H), 8.60 (d, J=5.0 Hz, 1H), 8.48 (s, 1H), 8.43 (d, J=5.0 Hz, 1H), 8.23 (d, J=5.0 Hz, 1H), 7.96 (d, J=15.0 Hz, 1H), 7.87 (d, J=5.0 Hz, 1H), 7.52-7.45 (m, 4H), 7.39-7.36 (m, 2H), 6.83 (d, J=5.0 Hz, 1H), 6.53 (d, J=10.0 Hz, 1H), 4.44 (s, 2H), 4.26 (qt, J=5.0H, 2H), 3.96-3.94 (m, 2H), 3.77 (t, J=10.0 Hz, 2H), 3.32-3.30 (m, 2H), 3.14 (qt, J=10.0 2H), 1.31 (t, J=5.0 Hz, 3H)

### Example 2: Synthesis of 4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1-phenyl-1,2-dihydropyridine-3-carboxamide hydrochloride

The title compound was synthesized by a method described in Example 1 of Korean Patent No. 10-2221689.

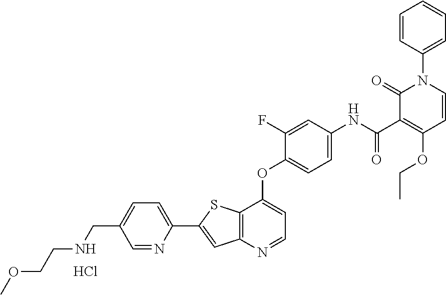 [SMILES: CCOc1ccn(-c2ccccc2)c(=O)c1C(=O)Nc1ccc(Oc2ccnc3cc(-c4ccc(CNCCOC)cn4)sc23)c(F)c1 ; Cl]

¹H NMR (500 MHz, DMSO-d₆) δ 10.70 (brs, 1H), 9.50 (brs, 2H), 8.80 (s, 1H), 8.69 (d, J=5.0 Hz, 1H), 8.47 (s, 1H), 8.44 (d, J=5.0 Hz, 1H), 8.22 (dd, J=10.0 and 5.0 Hz, 1H), 7.99 (d, J=10.0 Hz, 1H), 7.88 (d, J=5.0 Hz, 1H), 7.57-7.40 (m, 7H), 6.97 (d, J=5.0 Hz, 1H), 6.53 (d, J=5.0 Hz, 1H), 4.29-4.25 (m, 4H), 3.65 (t, J=5.0 Hz, 2H), 3.31 (s, 3H), 3.16-3.12 (m, 2H), 1.31 (t, J=5.0 Hz, 3H)

### Example 3: Synthesis of 4-ethoxy-N-{3-fluoro-4-[(2-{5-[(methylpiperazin-1-yl)methyl]pyridin-2-yl}thieno[3,2-b]pyridin-7-yl)oxy]phenyl}-2-oxo-1-phenyl-1,2-dihydropyridine-3-carboxamide hydrochloride

The title compound was synthesized by a method described in Example 34 of Korean Patent No. 10-2221689.

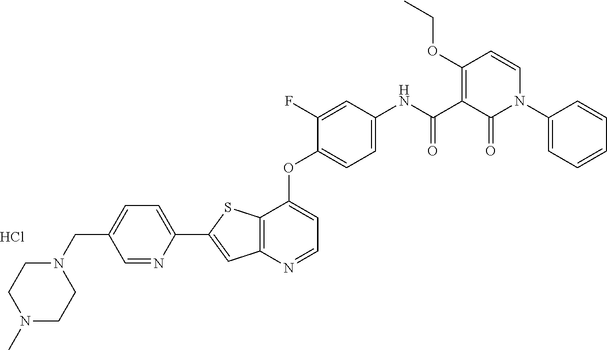 [SMILES: Cl ; CCOc1ccn(-c2ccccc2)c(=O)c1C(=O)Nc1ccc(Oc2ccnc3cc(-c4ccc(CN5CCN(C)CC5)cn4)sc23)c(F)c1]

¹H NMR (500 MHz, DMSO-d₆) δ 10.68 (s, 1H), 0.50 (brs, H), 8.80 (s, J, 8.80 (d, 1H), 8.47 (s, 1H), 8.41 (d, J=5.0 Hz, 1H), 8.23 (s, 1H), 7.97 (d, J=15.0 Hz, 1H), 7.87 (d, J=5.0 Hz, 1H), 7.56-7.46 (m, 5H), 7.42-7.40 (m, 2H), 6.86 (d, J=5.0 Hz, 1H), 6.52 (d, J=5.0 Hz, 1H), 4.26 (qt, J=5.0H, 2H), 3.93 (brs, 8H), 3.58 (brs, 2H), 2.81 (s, 3H), 1.31 (t, J=5.0 Hz, 3H)

### Example 4: Synthesis of N-(3-fluoro-4-((6-methoxy-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide

The title compound was synthesized by a method described in Example 78 of Korean Patent Laid-open Publication No. 10-2021-0015730.

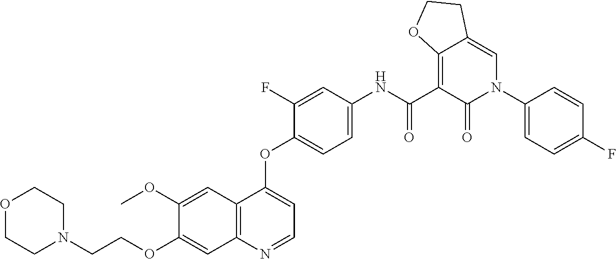 [SMILES: COc1cc2c(Oc3ccc(NC(=O)c4c5c(cn(-c6ccc(F)cc6)c4=O)CCO5)cc3F)ccnc2cc1OCCN1CCOCC1]

¹H NMR (500 MHz, DMSO-d₆) δ 11.89 (s, 1H), 8.48 (d, J=5.0 Hz, 1H), 8.00 (d, J=10.0 Hz, 1H), 7.88 (s, 1H), 7.54-7.49 (m, 3H), 7.45-7.37 (m, 5H), 6.48 (d, J=5.0 Hz, 1H), 4.85 (t, J=10.0 Hz, 2H), 4.32 (brs, 2H), 3.95 (s, 3H), 3.63 (brs, 4H), 3.11 (t, J=10.0 Hz, 2H), 2.52 (m, 2H, partially overlapped with DMSO), 2.50 (m, 4H, overlapped with DMSO)

### Example 5: Synthesis of N-(4-((7-(3-(3-cyanoazetidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide

The title compound was synthesized by a method described in Example 88 of Korean Patent Laid-open Publication No. 10-2021-0015730.

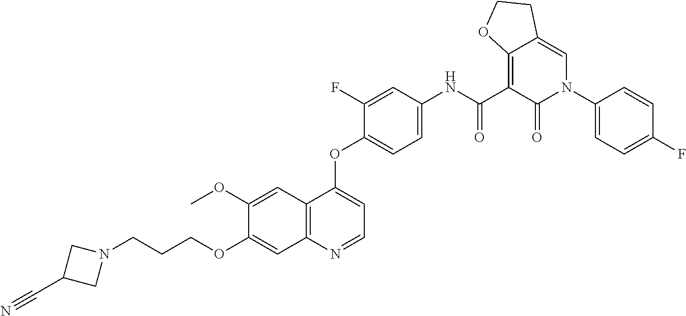 [SMILES: COc1cc2c(Oc3ccc(NC(=O)c4c5c(cn(-c6ccc(F)cc6)c4=O)CCO5)cc3F)ccnc2cc1OCCCN1CC(C#N)C1]

¹H NMR (400 MHz, CDCl₃) δ 12.04 (S, 1H), 8.48 (d, J=5.2 Hz, 1H), 8.03 (dd, J=12.4, 2.4 Hz, 1H), 7.57 (s, 1H), 7.40-7.37 (m, 5H), 7.26-7.22 (m, 2H), 7.17 (t, J=8.8 Hz, 1H), 6.44 (d, J=5.6 Hz, 1H), 5.01 (t, J=8.4 Hz, 2H), 4.26 (t, J=6.4 Hz, 2H), 4.03 (s, 3H), 3.64 (t, J=11.6 Hz, 4H), 3.20 (t, J=8.0 Hz, 2H), 2.81 (t, J=6.8 Hz, 2H), 2.05 (q, J=6.8 Hz, 3H)

### Positive Control 1

As a positive control 1 compound, N-{4-[(2-amino-3-chloro-4-pyridinyl)oxy]-3-fluorophenyl}-4-ethoxy-1-(4-fluorophenyl)-2-oxo-1,2-dihydro-3-pyridinecarboxamide, which is Compound 1 disclosed in U.S. Pat. No. 8,536,200 B2, was used, and this compound is a RON inhibitor well known as BMS-777607.

### Experimental Example 1. Identification of Apoptosis Efficacy Upon Inhibition of mtRON in Cholangiocarcinoma Cell Line (KKU-213: mRONΔ160)

To identify an effect of mRON in KKU-213 cell line, which is a cholangiocarcinoma cell line of mutant RONΔ160 type, shRNA was used to suppress expression, and then it was checked whether cell death was induced.

Specifically, the KKU-213 cell line (DMEM, manufacturer: Welgene, cat no: LM001-05, medium composition: 10% FBS, 1% penicillin/streptomycin), which is a cholangiocarcinoma cell line of mutant RONΔ160 type, was counted, seeded at 1×10⁵ cells per dish in a 60 mm cell culture dish (SPL, cat no: 20060), and cultured in a 5% CO₂ incubator at 37° C. After 24 hours, treatment with shRNA (scramble; AAUUCUCCGAACGUGUCACGU UCUC ACGUGACACGUUCGGAGAAUU UU (SEQ ID NO: 5)), shRON (exon13; CAGCUAGUUCUUCCUCCCAACCUGA UCUC UCAGGUUGGGAGGAAGAACUAGCUG UU (SEQ ID NO: 6)) at 200 pmol each was performed using Lipofectamine 2000 (Invitrogen, cat no: 11668019). After performing culture for 72 hours in a 5% CO₂ incubator at 37° C., all of the medium was transferred to a 15 ml conical tube (SPL, cat no: 50015), washing was performed once with 1×PBS (20×PBS, manufacturer: T&1, cat no: BPB-9121-010LB, diluted 20 times with distilled water and used after autoclaving), and then treatment with 1 ml of 0.05% trypsin-EDTA (Welgene, cat no: LS015-09) was performed. Thereafter, all cells were collected and centrifuged at 1500 rpm for 5 minutes. Then, the supernatant was removed. The cells were resuspended in 1×PBS and stained with trypan blue solution (Gibco, cat no: 15250061). The stained cells were counted using a hemocytometer. Cell counting was performed in duplicate, and the mean and standard deviation were calculated.

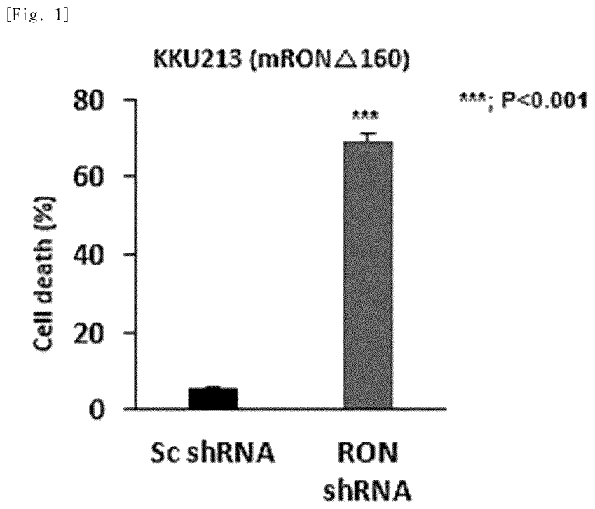

As a result, it was identified that the KKU-213 cell line treated with Sc shRNA was almost not killed, whereas the KKU-213 cell line treated with RON shRNA was killed by 70% or higher. From these results, it was identified that cell death is induced upon knockdown of RON in cholangiocarcinoma cells of mutant RON type (FIG. 1).

### Experimental Example 2. Confirmation of Efficacy of Inhibiting Cell Proliferation Depending on RON Genotype in Cholangiocarcinoma Cell Lines

KKU-213, which a cholangiocarcinoma cell line of mutant RONΔ160 type, KKK-D138, which is a cholangiocarcinoma cell line of mutant RONΔ155 type (DMEM, manufacturer: Welgene, cat no: LM001-05, medium composition: 10% FBS, 1% penicillin/streptomycin) was seeded in a 96-well-plate (SPL, cat no: 30096) and cultured in an incubator at a condition of 37° C. and 5% CO₂. Here, the KKU-213 and KKK-D138 cell lines were sequentially seeded at 1×10³ and 2×10³ cells/well, and the cell lines were purchased from JCRB. After 24 hours, WM-S1-030 (Example 1 to Example 5), and BMS-777607 (positive control 1) were diluted by 1/2 from 10 μM, and applied at 9 concentrations. In the negative control, DMSO (Dimethyl sulfoxide) was added as much as the amount of the drug diluted in the medium, and dispensing was performed.

In addition, Choi-CK, which is a cholangiocarcinoma cell line of mutant RONΔ165 type (DMEM, manufacturer: Welgene, cat no: LM001-05, medium composition: 10% FBS, 1% penicillin/streptomycin, 2 mM L-glutamine) was seeded at 2×10³ cells/well in a 96-well-plate (SPL, cat no: 30096), and cultured in an incubator at a condition of 37° C. and 5% CO₂. After 24 hours, WM-S1-030 (Example 1 to Example 5) and BMS-777607 (positive control 1) were diluted by 1/4 from 10 μM, and applied at 9 concentrations.

First, for MTS analysis, after drug treatment, culture was performed for 96 hours in an incubator at a condition of 37° C. and 5% CO₂, and the supernatant was removed. After dispensing fresh medium at 100 μl/well, MTS solution (Promega, cat no: G3582) was dispensed at 20 μl/well and cultured in an incubator at a condition of 37° C. and 5% CO₂. Using Victor X5 (PerkinElmer, Inc.), absorbance was measured at 490 nm on an hourly basis. A case where a ratio between the negative control and the positive control reached about 8 times or higher was determined to be optimal, and measurement was performed. From the obtained results, GraphPad prism 5 was used so that the x-axis represented drug concentration and the y-axis represented % relative cell viability, thereby producing results.

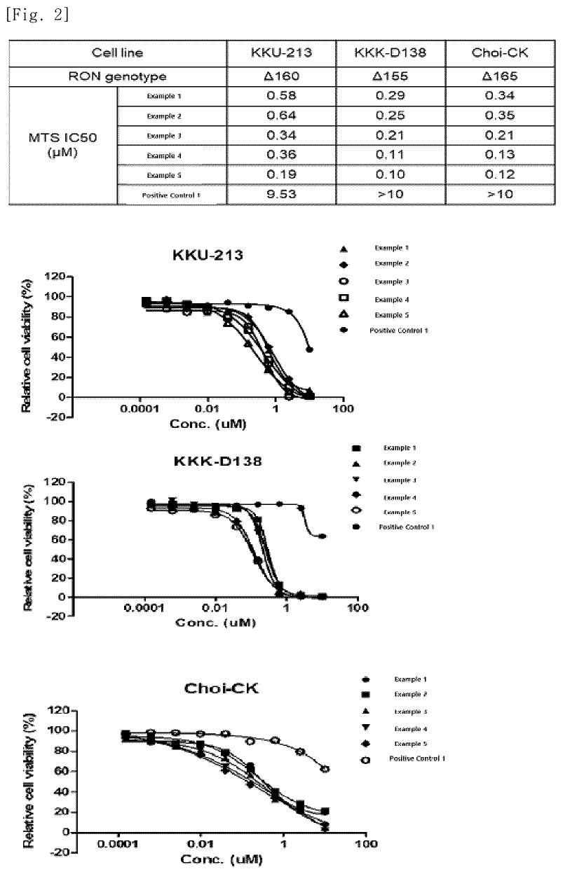

As a result, it was confirmed that cell proliferation was inhibited in a cholangiocarcinoma cell line of mutant RON type treated with Example 1 (WM-S1-030) to Example 5 compared to positive control 1 (BMS-777607) (FIG. 2).

### Experimental Example 3. Identification of Tumor Growth Inhibition Efficacy Caused by Example 1 in Animal Model Transplanted with Cholangiocarcinoma Cell Line (KKU-213: mRONΔ160) (I)

It was identified whether tumor growth was inhibited by administration of WM-S1-030 (Example 1) at doses of 30 mpk and 50 mpk to a mouse model transplanted with KKU-213 that is a cholangiocarcinoma cell line of mutant RONΔ160 type.

Specifically, 6-week-old female BALB/c-nude mice were obtained and acclimatized for 7 days. Then, the human cholangiocarcinoma cell line KKU-213 (2.5×10⁶ cells/mice) was diluted in PBS, and 100 μl of the dilution was subcutaneously injected into the right abdominal lateral side of the mice. When the tumor volume reached about 100 mm³, excipients and WM-S1-030 (Example 1) were orally administered. Here, as a positive control, BMS-777607 (positive control 1) was administered at a dose of 50 mpk. The drug was administered once a day for 3 weeks, and the tumor volume and body weight were measured twice a week. After the drug administration was completed, the mice were euthanized, and then the tumors were excised and weighed.

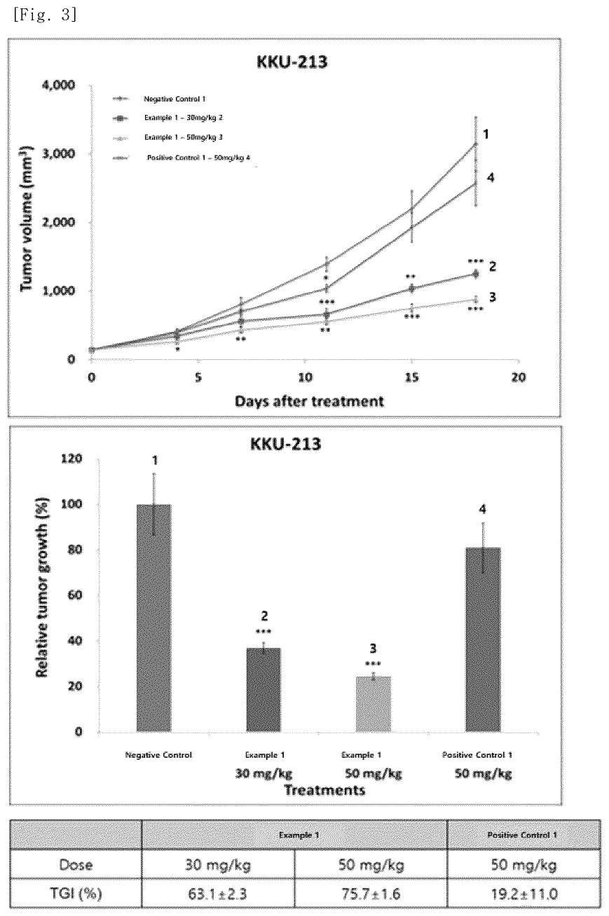

As a result, it was identified that the tumor growth was significantly inhibited, in proportion to the administered dose, in the mice administered with WM-S1-030 (Example 1) at doses of 30 mpk and 50 mpk. On the contrary, it was identified that BMS-777607 (positive control 1) exhibited relatively little tumor growth inhibition (FIG. 3).

Furthermore, Example 1 (WM-S1-030) at respective concentrations (30 mpk and 50 mpk) and positive control 1 (BMS-777607) at a dose of 50 mpk were administered to the cholangiocarcinoma cell line-transplanted mouse model, and then the tumors were enucleated and changes in expression of proteins were analyzed through immunochemical staining.

Specifically, paraffin slides were prepared using tissues isolated from the sacrificed mice. A deparaffinization process was performed twice for 5 minutes each using Histo-Clear (cat #HS-200), and an antigen recovery process was performed by immersion in citrate buffer (pH 6.0) (Cat #CBB999) and heating for 15 minutes. The slide was cooled sufficiently at room temperature, and then a blocking process was performed using Animal-Free Blocker® and Diluent (cat #SP-5035). pTyr-mRON antibody (anti-CD136 (RON) (pY1238/1239) antibody, #MBS 462024), mRON (anti RON antibody, #ab52927), Cleaved caspase 3 (C-cas3 (Asp175) antibody, MAB835), Cyclin D1 (CyclinD1 (SP4) antibody, #Z2027RS) antibodies were diluted to appropriate concentrations, and reaction was allowed to proceed overnight at 4° C. with care taken not to dry out. The next day, reaction was allowed to proceed at room temperature for 1 hour using the secondary antibody (VECTASTAIN Elite rabbit ABC HRP Kit (cat #PK-6101)). Then, DAB SUBSTRATE KIT (cat #SK-4100) was added, and time was allowed to elapse at room temperature until desired color development occurred. Then, the stained slide was treated with Richard-Allan Scientific Mounting Media, Non-Aqueous (cat #4112), a cover slide was placed thereon, and drying was performed for 1 hour or longer. Then, pictures were taken with a slide scanner (cat: M8, Precipoint).

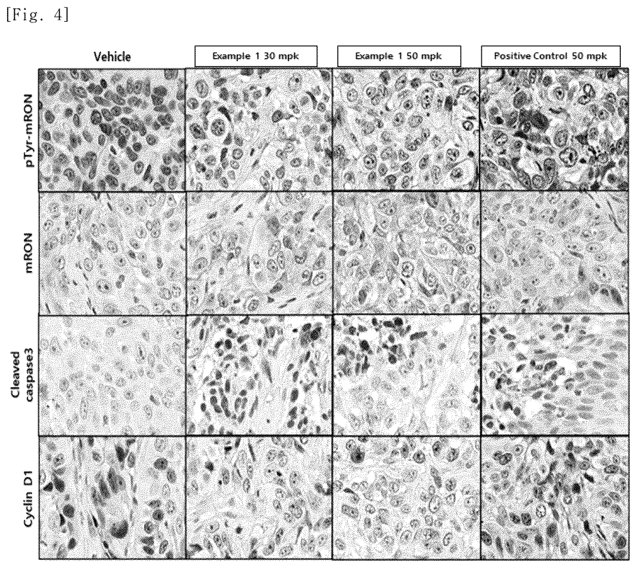

As a result, it was confirmed that in the group administered with Example 1 (WM-S1-030), the expression of pTyr-mRON was decreased, and the expression of cleaved caspase 3 was induced. In addition, it was confirmed that the expression of cyclin D1, which is a PD marker, was decreased (FIG. 4).

### Experimental Example 4. Identification of Tumor Growth Inhibition Efficacy Caused by Example 1 in Animal Model Transplanted with Cholangiocarcinoma Cell Line (Choi-CK: mRONΔ165) (II)

It was identified whether tumor growth was inhibited by administration of WM-S1-030 (Example 1) at doses of 30 mpk and 50 mpk to a mouse model transplanted with Choi-CK that is a cholangiocarcinoma cell line of mutant RONΔ165 type.

Specifically, 6-week-old female BALB/c-nude mice were obtained and acclimatized for 7 days. Then, the human cholangiocarcinoma cell line Choi-CK (5×10⁶ cells/mice) was diluted in PBS, and 100 μl of the dilution was subcutaneously injected into the right abdominal lateral side of the mice. When the tumor volume reached about 100 mm³, excipients and WM-S1-030 (Example 1) were orally administered. Here, as a positive control, BMS-777607 (positive control 1) was administered at a dose of 50 mpk. The drug was administered once a day for 3 weeks, and the tumor volume and body weight were measured twice a week. After the drug administration was completed, the mice were euthanized, and then the tumors were excised and weighed.

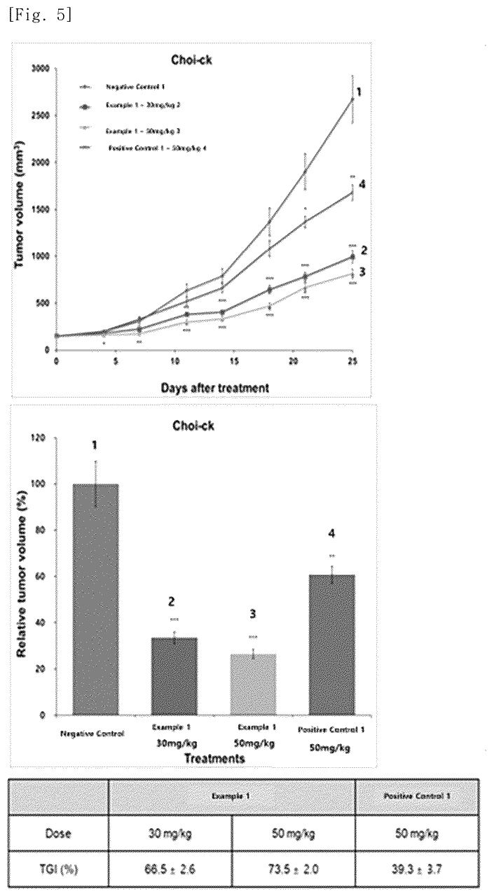

As a result, it was identified that the tumor growth was significantly inhibited, in proportion to the administered dose, in the mice administered with WM-S1-030 (Example 1) at doses of 30 mpk and 50 mpk. On the contrary, it was identified that BMS-777607 (positive control 1) exhibited relatively little tumor growth inhibition (FIG. 5).

Furthermore, WM-S1-030 (Example 1) at respective concentrations (30 mpk, 50 mpk) and BMS-777607 (positive control 1) at a dose of 50 mpk were administered to the cholangiocarcinoma cell line-transplanted mouse model, and then the tumors were excised and analyzed for changes in expression of proteins through immunochemical staining.

Specifically, paraffin slides were prepared using tissues isolated from the sacrificed mice. A deparaffinization process was performed twice for 5 minutes each using Histo-Clear (cat #HS-200), and an antigen recovery process was performed by immersion in citrate buffer (pH 6.0) (Cat #CBB999) and heating for 15 minutes. The slide was cooled sufficiently at room temperature, and then a blocking process was performed using Animal-Free Blocker® and Diluent (cat #SP-5035). pTyr-mRON antibody (anti-CD136 (RON) (pY1238/1239) antibody, #MBS 462024), mRON (anti RON antibody, #ab52927), Cleaved caspase 3 (C-cas3 (Asp175) antibody, MAB835), Cyclin D1 (CyclinD1 (SP4) antibody, #Z2027RS) antibodies were diluted to appropriate concentrations, and reaction was allowed to proceed overnight at 4° C. with care taken not to dry out. The next day, reaction was allowed to proceed at room temperature for 1 hour using the secondary antibody (VECTASTAIN Elite rabbit ABC HRP Kit (cat #PK-6101)). Then, DAB SUBSTRATE KIT (cat #SK-4100) was added, and time was allowed to elapse at room temperature until desired color development occurred. Then, the stained slide was treated with Richard-Allan Scientific Mounting Media, Non-Aqueous (cat #4112), a cover slide was placed thereon, and drying was performed for 1 hour or longer. Then, pictures were taken with a slide scanner (cat: M8, Precipoint).

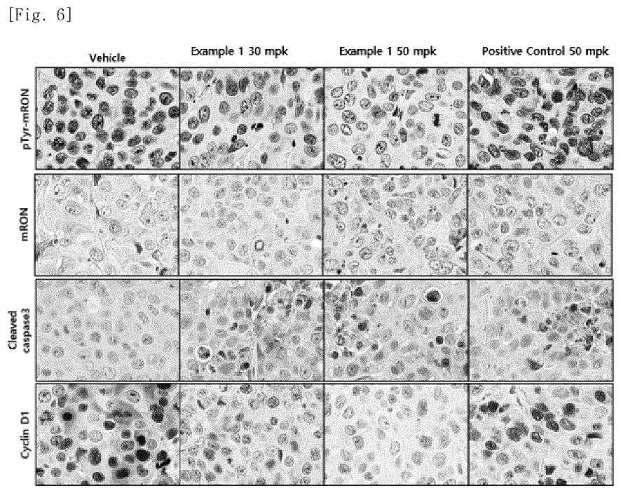

As a result, it was identified that in the group administered with WM-S1-030 (Example 1), the expression of pTyr-mRON was decreased, and the expression of cleaved caspase 3 was induced. In addition, it was identified that the expression of cyclin D1, which is a PD marker, was decreased (FIG. 6).

### Experimental Example 5. Confirmation III of Efficacy of Inhibiting Tumor Growth by Example 1 in Animal Model Transplanted with Cholangiocarcinoma Patient-Derived Cancer Tissue (CTG-0927: mRON Δ165)

It was confirmed whether tumor growth was inhibited by the administration of Example 1 (WM-S1-030) at doses of 10 mpk or 30 mpk to a mouse model transplanted with CTG-0927 that is a cholangiocarcinoma patient-derived cancer tissue of mutant RONΔ165 type.

Specifically, 6-to-8-week-old female athymic nude-Foxn1nu mice were transplanted at the left ventral side thereof with CTG-0927 that is a cholangiocarcinoma patient-derived cancer tissue. Example 1 (WM-S1-030) was orally administered when the tumor reached a size of about 50-150 mm³. The drug was administered once a day for 8 weeks, and tumor size and body weight were measured twice a week. After the drug administration was completed, the mice were euthanized, and then the tumors were enucleated and weighed.

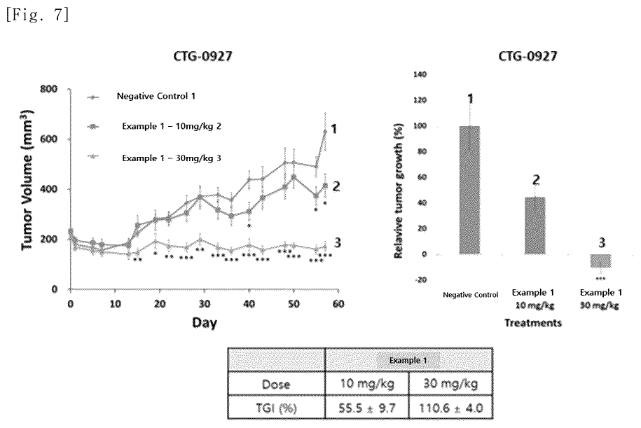

As a result, it was confirmed that the tumor growth was significantly inhibited, in proportion to the administered dose, in the mice administered with Example 1 (WM-S1-030) at doses of 10 mpk and 30 mpk (FIG. 7).

### Experimental Example 6. Analysis of RON Mutations in Cholangiocarcinoma Cell Lines

For 14 cholangiocarcinoma cell lines, total RNA was extracted using High Pure RNA Isolation Kit (Roche, Cat #11828665001) according to the manufacturer's manual. Then, a total volume of 20 μl of cDNA was synthesized from 1 μg of total RNA using oligo (dT). Using 2 μl out of this volume, RT-PCR was performed for analysis of RON variants.

For deletion of RON Exons 5 & 6, PCR was performed using primers (forward: 5′-GAGCTGGTCAGGTCACTAAAC-3′ (SEQ ID NO: 6), reverse: 5′-CAGACACTCAGTCCCATTGAC-3′ (SEQ ID NO: 7)) under the condition of a total of 35 cycles of denaturation at 95° C. for 30 seconds/annealing at 59° C. for 30 seconds/extension at 72° C. for 45 seconds. For deletion of RON Exon 11, PCR was performed using primers (forward: 5′-ATCTGTGGCCAGCATCTAAC-3′ (SEQ ID NO: 8), reverse: 5′-AAAGGCAGCAGGATACCAAG-3′ (SEQ ID NO: 9)) under the same condition as described above. As an internal control, GAPDH, which is a housekeeping gene, was used. The PCR product was extracted using the QIA quick PCR purification kit (Qiagen, cat #28106), and a request for sequencing thereof was made to Cosmogenetech, Co., Ltd. The sequencing results were analyzed to determine the RON mutation and mutation type.

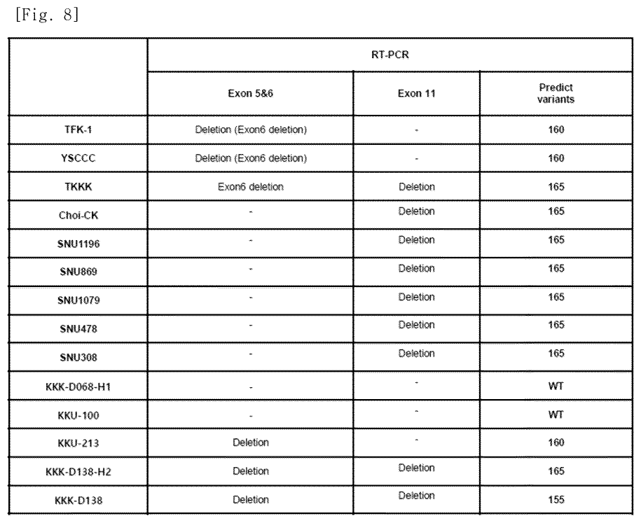

As a result, in most of the cholangiocarcinoma cell lines, the RON mutation was identified and a high proportion of mutation RONΔ165 with exon 11 deleted was identified (FIG. 8).

### Experimental Example 7. Analysis of RON Genotype in Cholangiocarcinoma Patient Tissue

For 125 cholangiocarcinoma patient tissues, total RNA was extracted using High Pure RNA Tissue Kit (Roche, Cat #12033674001) according to the manufacturer's manual. A total volume of 20 μl of cDNA was synthesized from 1 μg of total RNA using oligo (dT). Using 2 μl out of this volume, RT-PCR was performed for analysis of RON variants. Here, RT-PCR was performed in the same manner as in Experimental Example 6.

The PCR product was extracted using the QIA quick PCR purification kit (Qiagen, cat #28106), and a request for sequencing thereof was made to Cosmogenetech, Co., Ltd. The sequencing results were analyzed to determine the RON mutation and mutation type.

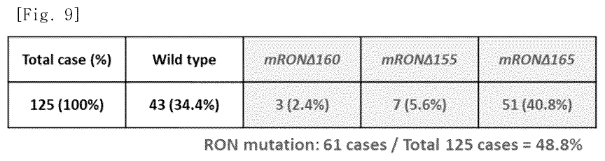

As a result, the RON mutation was identified in about 48.8% of the cholangiocarcinoma patient tissues. All three types of mutations RONΔ155, RONΔ160, and RONΔ165 were identified, and among these, the mutation RONΔ165 with exon 11 deleted was identified at a high proportion (FIG. 9).

1. A method for preventing or treating cholangiocarcinoma in a subject in need thereof, comprising administering to the subject an effective amount of a pharmaceutical composition comprising, as an active ingredient, a compound of Formula 1 or Formula 2 below, or a pharmaceutically acceptable salt thereof: 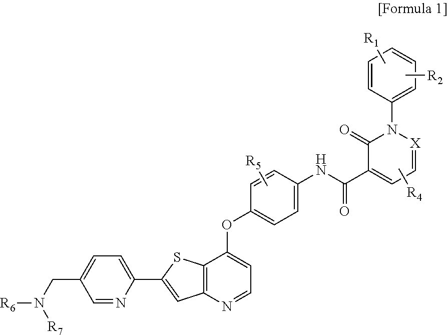
wherein, in Formula 1 above,
R₁ and R₂ are each independently H, halogen, C₁₋₁₀ alkoxy, or halo C₁₋₁₀ alkyl;
X is —C(—R₃)═ or —N═;
R₃ and R₄ are each independently H, halogen, C₁₋₁₀ alkyl, or C₁₋₁₀ alkoxy;
R₅ is H, halogen, or C₁₋₁₀ alkyl;
R₆ and R₇ form a 4- to 10-membered heterocycle together with the N atom to which they are bonded, or R₆ is —C₂H₄O—CH₃, and R₇ is H, methyl, or t-butoxycarbonyl; and
the heterocycle optionally further has one or two heteroatoms selected from the group consisting of N, O, and S, in addition to the N atom to which R₆ and R₇ are bonded, and is unsubstituted or substituted with one or more substituents selected from among halogen and C₁₋₆ alkyl, 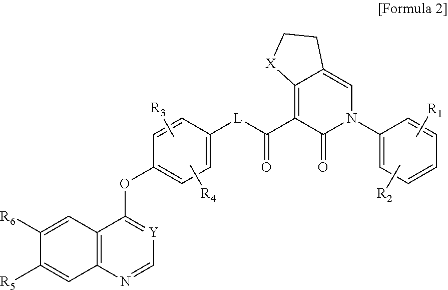
wherein, in Formula 2 above,
L is —NH— or —CH₂—,
R₁ to R₄ are each independently hydrogen, halogen, hydroxy, cyano, C₁₋₄ alkyl, C₁₋₄ alkoxy, C₁₋₄ haloalkyl, C₂₋₄ alkenyl, C₂₋₄ alkynyl, C₃₋₇ cycloalkyl, C₆₋₁₀ aryl, 5- to 9-membered heteroaryl, or 3- to 9-membered heterocycloalkyl,
X is O, S, —CH(-Rx)-, or —N(-Rx)-,
Rx is hydrogen, C₁₋₄ alkyl, C₁₋₄ alkoxy, C₁₋₄ haloalkyl, C₂₋₄ alkenyl, C₂₋₄ alkynyl, C₆₋₁₀ aryl, C₆₋₁₀ aryl-C₁₋₄ alkyl, or 3- to 9-membered heterocycloalkyl,
Y is —N═ or —CH═, and
R₅ and R₆ are each independently hydrogen, amino, halogen, cyano, C₁₋₆ alkyl, C₁₋₆ haloalkyl, C₁₋₆ alkoxy, amino-C₁₋₆ alkoxy, aminocarbonyl, C₁₋₆ alkylaminocarbonyl, diC₁₋₆ alkylcarbonylamino, C₁₋₆ alkylcarbonylamino, C₁₋₆ alkylamino, or C₁₋₆ alkyl-amino-C₁₋₆ alkoxy, wherein R₅ and R₆ are each independently unsubstituted or substituted with 3- to 9-membered cycloalkyl or 3- to 9-membered heterocycloalkyl,
the cycloalkyl or the heterocycloalkyl optionally has one or more substituents selected from the group consisting of halogen, oxo, cyano, hydroxy, hydroxy-C₁₋₆ alkyl, amino, diC₁₋₆ alkylamino, C₁₋₆ alkyl, C₁₋₆ alkoxy, and C₁₋₆ alkoxy-C₁₋₆ alkyl, and
the heterocycloalkyl contains 1 to 4 heteroatoms selected from the group consisting of N, O, and S,
wherein the cholangiocarcinoma has a recepteur d'origine nantais (RON) mutation.

2. The method of claim 1, wherein the compound of Formula 1 is a compound selected from the group consisting of:
4-ethoxy-N-[3-fluoro-4-({2-[5-(morpholinomethyl)pyridin-2-yl]thieno[3,2-b]pyridin-7-yl}oxy)phenyl]-1-(4-fluorophenyl)-2-oxo-1,2-dihydropyridine-3-carboxamide;
4-ethoxy-N-(3-fluoro-4-{[2-(5-{[(2-methoxyethyl)amino]methyl}pyridin-2-yl)thieno[3,2-b]pyridin-7-yl]oxy}phenyl)-2-oxo-1-phenyl-1,2-dihydropyridine-3-carboxamide; and
4-ethoxy-N-(3-fluoro-4-[(2-{5-[(methylpiperazin-1-yl)methyl]pyridin-2-yl}thieno[3,2-b]pyridin-7-yl)oxy]phenyl}-2-oxo-1-phenyl-1,2-dihydropyridine-3-carboxamide.

3. The method of claim 1, wherein the compound of Formula 2 is N-(3-fluoro-4-((6-methoxy-7-(2-morpholinoethoxy)quinolin-4-yl)oxy)phenyl)-5-(4-fluorophenyl)-6-oxo-2,3,5,6-tetrahydrofuro[3,2-c]pyridine-7-carboxamide; or N-(4-((7-(3-(3-cyanoazetidin-1-yl)propoxy)-6-methoxyquinolin-4-yl)oxy)-3-fluorophenyl)-5-(4-fluorophenyl)-6-oxo-2,3,5,6-tetrahydrofuro[3,2-c]pyridine-7-carboxamide.

4. (canceled)

5. The method of claim 1, wherein the chonagiocarcinoma exhibits resistance to an EGFR-targeted therapeutic agent.

6. The method of claim 1, wherein the RON mutation is RONΔ155 in which exons 5, 6, and 11 are deleted, RONΔ160 in which exons 5 and 6 are deleted, or RONΔ165 in which exon 11 is deleted.

7. The method of claim 5, wherein the EGFR-targeted therapeutic agent is at least one selected from the group consisting of cetuximab, gefitinib, erlotinib, apatinib, icotinib, brigatinib, lapatinib, canertinib, AEE788, XL647, zactima, panitumumab, and a combination thereof.

8. The method of claim 1, which further the step of detecting RON mutation in a biological sample of the subject, wherein the RON mutation is RONΔ155 in which exons 5, 6, and 11 are deleted, RONΔ160 in which exons 5 and 6 are deleted, or RONΔ165 in which exon 11 is deleted.

9.-11. (canceled)


SEQUENCE LISTING (9 sequences)

SEQ ID NO: 1 - DNA, 3732 bp, Artificial Sequence
  RON 155 cDNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtgtacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgaggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggcagtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 2 - DNA, 3879 bp, Artificial Sequence
  RON 160 cDNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtgtacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgaggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggcagtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 3 - DNA, 4056 bp, Artificial Sequence
  RON 165 cDNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 4 - RNA, 48 nt, Artificial Sequence
  Sc shRNA
aauucuccgaacgugucacguucucacgugacacguucggagaauuuu

SEQ ID NO: 5 - RNA, 56 nt, Artificial Sequence
  RON shRNA
cagcuaguucuuccucccaaccugaucucucagguugggaggaagaacuagcuguu

SEQ ID NO: 6 - DNA, 21 bp, Artificial Sequence
  RON d5_6 forward primer
gagctggtcaggtcactaaac

SEQ ID NO: 7 - DNA, 21 bp, Artificial Sequence
  RON d5_6 reverse primer
cagacactcagtcccattgac

SEQ ID NO: 8 - DNA, 20 bp, Artificial Sequence
  RON d11 forward primer
atctgtggccagcatctaac

SEQ ID NO: 9 - DNA, 20 bp, Artificial Sequence
  RON d11 reverse primer
aaaggcagcaggataccaag